SEQUENCE LISTING (167 sequences)

SEQ ID NO: 1 - protein, 33 aa, Artificial Sequence
  Synthetic polypeptide
RMDRIGLSKGCFGLKLDRIGSMSGLGCKVLRRH

SEQ ID NO: 2 - protein, 5 aa, Homo sapiens
RMDRI

SEQ ID NO: 3 - protein, 22 aa, Homo sapiens
GLSKGCFGLKLDRIGSMSGLGC

SEQ ID NO: 4 - protein, 6 aa, Homo sapiens
KVLRRH

SEQ ID NO: 5 - protein, 33 aa, Artificial Sequence
  Synthetic polypeptide
RMDRIGLSKGCFGLKLDRIREASGLGCKVLRRH

SEQ ID NO: 6 - protein, 22 aa, Artificial Sequence
  Synthetic polypeptide
GLSKGCFGLKLDRIREASGLGC

SEQ ID NO: 7 - protein, 28 aa, Homo sapiens
SLRRSSCFGGRMDRIGAQSGLGCNSFRY

SEQ ID NO: 8 - protein, 32 aa, Homo sapiens
SPKMVQGSGCFGRKMDRISSSSGLGCKVLRRH

SEQ ID NO: 9 - protein, 32 aa, Homo sapiens
TAPRSLRRSSCFGGRMDRIGAQSGLGCNSFRY

SEQ ID NO: 10 - protein, 38 aa, Dendroaspis
EVKYDPCFGHKIDRINHVSNLGCPSLRDPRPNAPSTSA

SEQ ID NO: 11 - protein, 28 aa, Artificial Sequence
  Synthetic polypeptide
SLRRSSCFGGRMDRIXXXSGLGCNSFRY

SEQ ID NO: 12 - protein, 28 aa, Artificial Sequence
  Synthetic polypeptide
SLRRSSCFGGRMDRIREASGLGCNSFRY

SEQ ID NO: 13 - protein, 32 aa, Artificial Sequence
  Synthetic polypeptide
SPKMVQGSGCFGRKMDRIXXXSGLGCKVLRRH

SEQ ID NO: 14 - protein, 32 aa, Artificial Sequence
  Synthetic polypeptide
SPKMVQGSGCFGRKMDRIREASGLGCKVLRRH

SEQ ID NO: 15 - protein, 22 aa, Artificial Sequence
  Synthetic polypeptide
GLSKGCFGLKLDRIXXXSGLGC

SEQ ID NO: 16 - protein, 32 aa, Artificial Sequence
  Synthetic polypeptide
TAPRSLRRSSCFGGRMDRIXXXSGLGCNSFRY

SEQ ID NO: 17 - protein, 32 aa, Artificial Sequence
  Synthetic polypeptide
TAPRSLRRSSCFGGRMDRIREASGLGCNSFRY

SEQ ID NO: 18 - protein, 38 aa, Artificial Sequence
  Synthetic polypeptide
EVKYDPCFGHKIDRIXXXSNLGCPSLRDPRPNAPSTSA

SEQ ID NO: 19 - protein, 38 aa, Artificial Sequence
  Synthetic polypeptide
EVKYDPCFGHKIDRIREASNLGCPSLRDPRPNAPSTSA

SEQ ID NO: 20 - protein, 26 aa, Artificial Sequence
  Synthetic polypeptide
SPKMVQGSGCFGRKMDRIXXXSGLGC

SEQ ID NO: 21 - protein, 15 aa, Dendroaspis
PSLRDPRPNAPSTSA

SEQ ID NO: 22 - protein, 41 aa, Artificial Sequence
  Synthetic polypeptide
SPKMVQGSGCFGRKMDRIXXXSGLGCPSLRDPRPNAPSTSA

SEQ ID NO: 23 - protein, 41 aa, Artificial Sequence
  Synthetic polypeptide
SPKMVQGSGCFGRKMDRIREASGLGCPSLRDPRPNAPSTSA

SEQ ID NO: 24 - protein, 37 aa, Artificial Sequence
  Synthetic polypeptide
GLSKGCFGLKLDRIXXXSGLGCPSLRDPRPNAPSTSA

SEQ ID NO: 25 - protein, 37 aa, Artificial Sequence
  Synthetic polypeptide
GLSKGCFGLKLDRIREASGLGCPSLRDPRPNAPSTSA

SEQ ID NO: 26 - protein, 10 aa, Homo sapiens
TAPRSLRRSS

SEQ ID NO: 27 - protein, 17 aa, Artificial Sequence
  Synthetic polypeptide
CFGLKLDRIXXXSGLGC

SEQ ID NO: 28 - protein, 5 aa, Homo sapiens
NSFRY

SEQ ID NO: 29 - protein, 32 aa, Artificial Sequence
  Synthetic polypeptide
TAPRSLRRSSCFGLKLDRIXXXSGLGCNSFRY

SEQ ID NO: 30 - protein, 32 aa, Artificial Sequence
  Synthetic polypeptide
TAPRSLRRSSCFGLKLDRIREASGLGCNSFRY

SEQ ID NO: 31 - protein, 6 aa, homo sapiens
SLRRSS

SEQ ID NO: 32 - protein, 17 aa, Artificial Sequence
  Synthetic polypeptide
CFGRKMDRIXXXSGLGC

SEQ ID NO: 33 - protein, 28 aa, Artificial Sequence
  Synthetic polypeptide
SLRRSSCFGRKMDRIXXXSGLGCNSFRY

SEQ ID NO: 34 - protein, 28 aa, Artificial Sequence
  Synthetic polypeptide
SLRRSSCFGRKMDRIREASGLGCNSFRY

SEQ ID NO: 35 - protein, 32 aa, Artificial Sequence
  Synthetic polypeptide
TAPRSLRRSSCFGRKMDRIXXXSGLGCNSFRY

SEQ ID NO: 36 - protein, 32 aa, Artificial Sequence
  Synthetic polypeptide
TAPRSLRRSSCFGRKMDRIREASGLGCNSFRY

SEQ ID NO: 37 - protein, 28 aa, Artificial Sequence
  Synthetic polypeptide
SLRRSSCFGLKLDRIXXXSGLGCNSFRY

SEQ ID NO: 38 - protein, 28 aa, Artificial Sequence
  Synthetic polypeptide
SLRRSSCFGLKLDRIREASGLGCNSFRY

SEQ ID NO: 39 - protein, 29 aa, Artificial Sequence
  Synthetic polypeptide
SLRRSSCFGLKLDRIXXXSGLGCKVLRRH

SEQ ID NO: 40 - protein, 29 aa, Artificial Sequence
  Synthetic polypeptide
SLRRSSCFGLKLDRIREASGLGCKVLRRH

SEQ ID NO: 41 - protein, 9 aa, Homo sapiens
SPKMVQGSG

SEQ ID NO: 42 - protein, 32 aa, Artificial Sequence
  Synthetic polypeptide
SPKMVQGSGCFGLKLDRIXXXSGLGCKVLRRH

SEQ ID NO: 43 - protein, 32 aa, Artificial Sequence
  Synthetic polypeptide
SPKMVQGSGCFGLKLDRIREASGLGCKVLRRH

SEQ ID NO: 44 - protein, 6 aa, Dendroaspis
EVKYDP

SEQ ID NO: 45 - protein, 38 aa, Artificial Sequence
  Synthetic polypeptide
EVKYDPCFGLKLDRIXXXSGLGCPSLRDPRPNAPSTSA

SEQ ID NO: 46 - protein, 38 aa, Artificial Sequence
  Synthetic polypeptide
EVKYDPCFGLKLDRIREASGLGCPSLRDPRPNAPSTSA

SEQ ID NO: 47 - protein, 33 aa, Artificial Sequence
  Synthetic polypeptide
TAPRSLRRSSCFGLKLDRIXXXSGLGCKVLRRH

SEQ ID NO: 48 - protein, 33 aa, Artificial Sequence
  Synthetic polypeptide
TAPRSLRRSSCFGLKLDRIREASGLGCKVLRRH

SEQ ID NO: 49 - protein, 33 aa, Artificial Sequence
  Synthetic polypeptide
RMDRIGLSKGCFGLKLDRIXXXSGLGCKVLRRH

SEQ ID NO: 50 - protein, 28 aa, Artificial Sequence
  Synthetic polypeptide
GLSKGCFGLKLDRIXXXSGLGCKVLRRH

SEQ ID NO: 51 - protein, 28 aa, Artificial Sequence
  Synthetic polypeptide
GLSKGCFGLKLDRIREASGLGCKVLRRH

SEQ ID NO: 52 - protein, 28 aa, Artificial Sequence
  Synthetic polypeptide
TLRRSSCFGGRMDRIREASGLGCNSFRY

SEQ ID NO: 53 - protein, 28 aa, Artificial Sequence
  Synthetic polypeptide
SIRRSSCFGGRMDRIREASGLGCNSFRY

SEQ ID NO: 54 - protein, 28 aa, Artificial Sequence
  Synthetic polypeptide
SLKRSSCFGGRMDRIREASGLGCNSFRY

SEQ ID NO: 55 - protein, 28 aa, Artificial Sequence
  Synthetic polypeptide
SLRKSSCFGGRMDRIREASGLGCNSFRY

SEQ ID NO: 56 - protein, 28 aa, Artificial Sequence
  Synthetic polypeptide
SLRRSSCFGGRMDRIREASGLGCNTFRY

SEQ ID NO: 57 - protein, 28 aa, Artificial Sequence
  Synthetic polypeptide
SLRRSSCFGGRMDRIREASGLGCNSLRY

SEQ ID NO: 58 - protein, 28 aa, Artificial Sequence
  Synthetic polypeptide
SLRRSSCFGGRMDRIREASGLGCNSFKY

SEQ ID NO: 59 - protein, 28 aa, Artificial Sequence
  Synthetic polypeptide
SLRRSSCFGGRMDRIREASGLGCNSFRF

SEQ ID NO: 60 - protein, 32 aa, Artificial Sequence
  Synthetic polypeptide
TPKMVQGSGCFGRKMDRIREASGLGCKVLRRH

SEQ ID NO: 61 - protein, 32 aa, Artificial Sequence
  Synthetic polypeptide
SGKMVQGSGCFGRKMDRIREASGLGCKVLRRH

SEQ ID NO: 62 - protein, 32 aa, Artificial Sequence
  Synthetic polypeptide
SPRMVQGSGCFGRKMDRIREASGLGCKVLRRH

SEQ ID NO: 63 - protein, 32 aa, Artificial Sequence
  Synthetic polypeptide
SPKLVQGSGCFGRKMDRIREASGLGCKVLRRH

SEQ ID NO: 64 - protein, 32 aa, Artificial Sequence
  Synthetic polypeptide
SPKMVQGSGCFGRKMDRIREASGLGCKVIRRH

SEQ ID NO: 65 - protein, 32 aa, Artificial Sequence
  Synthetic polypeptide
SPKMVQGSGCFGRKMDRIREASGLGCKVLKRH

SEQ ID NO: 66 - protein, 32 aa, Artificial Sequence
  Synthetic polypeptide
SPKMVQGSGCFGRKMDRIREASGLGCKVLRKH

SEQ ID NO: 67 - protein, 32 aa, Artificial Sequence
  Synthetic polypeptide
SPKMVQGSGCFGRKMDRIREASGLGCKVLRRR

SEQ ID NO: 68 - protein, 22 aa, Artificial Sequence
  Synthetic polypeptide
PLSKGCFGLKLDRIREASGLGC

SEQ ID NO: 69 - protein, 22 aa, Artificial Sequence
  Synthetic polypeptide
GISKGCFGLKLDRIREASGLGC

SEQ ID NO: 70 - protein, 22 aa, Artificial Sequence
  Synthetic polypeptide
GLTKGCFGLKLDRIREASGLGC

SEQ ID NO: 71 - protein, 22 aa, Artificial Sequence
  Synthetic polypeptide
GLSRGCFGLKLDRIREASGLGC

SEQ ID NO: 72 - protein, 22 aa, Artificial Sequence
  Synthetic polypeptide
GLSKGCFGLKLDRIREASPLGC

SEQ ID NO: 73 - protein, 22 aa, Artificial Sequence
  Synthetic polypeptide
GLSKGCFGLKLDRIREASGIGC

SEQ ID NO: 74 - protein, 22 aa, Artificial Sequence
  Synthetic polypeptide
GLSKGCFGLKLDRIREASGLPC

SEQ ID NO: 75 - protein, 22 aa, Artificial Sequence
  Synthetic polypeptide
GLSKGCFGLKLDRIREASGLGS

SEQ ID NO: 76 - protein, 41 aa, Artificial Sequence
  Synthetic polypeptide
TPKMVQGSGCFGRKMDRIREASGLGCPSLRDPRPNAPSTSA

SEQ ID NO: 77 - protein, 41 aa, Artificial Sequence
  Synthetic polypeptide
SGKMVQGSGCFGRKMDRIREASGLGCPSLRDPRPNAPSTSA

SEQ ID NO: 78 - protein, 41 aa, Artificial Sequence
  Synthetic polypeptide
SPRMVQGSGCFGRKMDRIREASGLGCPSLRDPRPNAPSTSA

SEQ ID NO: 79 - protein, 41 aa, Artificial Sequence
  Synthetic polypeptide
SPKLVQGSGCFGRKMDRIREASGLGCPSLRDPRPNAPSTSA

SEQ ID NO: 80 - protein, 41 aa, Artificial Sequence
  Synthetic polypeptide
SPKMVQGSGCFGRKMDRIREASGLGCPSLRDPRPNAPTTSA

SEQ ID NO: 81 - protein, 41 aa, Artificial Sequence
  Synthetic polypeptide
SPKMVQGSGCFGRKMDRIREASGLGCPSLRDPRPNAPSSSA

SEQ ID NO: 82 - protein, 41 aa, Artificial Sequence
  Synthetic polypeptide
SPKMVQGSGCFGRKMDRIREASGLGCPSLRDPRPNAPSTTA

SEQ ID NO: 83 - protein, 41 aa, Artificial Sequence
  Synthetic polypeptide
SPKMVQGSGCFGRKMDRIREASGLGCPSLRDPRPNAPSTSV

SEQ ID NO: 84 - protein, 37 aa, Artificial Sequence
  Synthetic polypeptide
PLSKGCFGLKLDRIREASGLGCPSLRDPRPNAPSTSA

SEQ ID NO: 85 - protein, 37 aa, Artificial Sequence
  Synthetic polypeptide
GISKGCFGLKLDRIREASGLGCPSLRDPRPNAPSTSA

SEQ ID NO: 86 - protein, 37 aa, Artificial Sequence
  Synthetic polypeptide
GLTKGCFGLKLDRIREASGLGCPSLRDPRPNAPSTSA

SEQ ID NO: 87 - protein, 37 aa, Artificial Sequence
  Synthetic polypeptide
GLSRGCFGLKLDRIREASGLGCPSLRDPRPNAPSTSA

SEQ ID NO: 88 - protein, 37 aa, Artificial Sequence
  Synthetic polypeptide
GLSKGCFGLKLDRIREASGLGCPSLRDPRPNAPTTSA

SEQ ID NO: 89 - protein, 37 aa, Artificial Sequence
  Synthetic polypeptide
GLSKGCFGLKLDRIREASGLGCPSLRDPRPNAPSSSA

SEQ ID NO: 90 - protein, 37 aa, Artificial Sequence
  Synthetic polypeptide
GLSKGCFGLKLDRIREASGLGCPSLRDPRPNAPSTTA

SEQ ID NO: 91 - protein, 37 aa, Artificial Sequence
  Synthetic polypeptide
GLSKGCFGLKLDRIREASGLGCPSLRDPRPNAPSTSV

SEQ ID NO: 92 - protein, 32 aa, Artificial Sequence
  Synthetic polypeptide
SAPRSLRRSSCFGLKLDRIREASGLGCNSFRY

SEQ ID NO: 93 - protein, 32 aa, Artificial Sequence
  Synthetic polypeptide
TVPRSLRRSSCFGLKLDRIREASGLGCNSFRY

SEQ ID NO: 94 - protein, 32 aa, Artificial Sequence
  Synthetic polypeptide
TAGRSLRRSSCFGLKLDRIREASGLGCNSFRY

SEQ ID NO: 95 - protein, 32 aa, Artificial Sequence
  Synthetic polypeptide
TAPKSLRRSSCFGLKLDRIREASGLGCNSFRY

SEQ ID NO: 96 - protein, 32 aa, Artificial Sequence
  Synthetic polypeptide
TAPRSLRRSSCFGLKLDRIREASGLGCNTFRY

SEQ ID NO: 97 - protein, 32 aa, Artificial Sequence
  Synthetic polypeptide
TAPRSLRRSSCFGLKLDRIREASGLGCNSLRY

SEQ ID NO: 98 - protein, 32 aa, Artificial Sequence
  Synthetic polypeptide
TAPRSLRRSSCFGLKLDRIREASGLGCNSFKY

SEQ ID NO: 99 - protein, 32 aa, Artificial Sequence
  Synthetic polypeptide
TAPRSLRRSSCFGLKLDRIREASGLGCNSFRF

SEQ ID NO: 100 - protein, 28 aa, Artificial Sequence
  Synthetic polypeptide
TLRRSSCFGRKMDRIREASGLGCNSFRY

SEQ ID NO: 101 - protein, 28 aa, Artificial Sequence
  Synthetic polypeptide
SIRRSSCFGRKMDRIREASGLGCNSFRY

SEQ ID NO: 102 - protein, 28 aa, Artificial Sequence
  Synthetic polypeptide
SLKRSSCFGRKMDRIREASGLGCNSFRY

SEQ ID NO: 103 - protein, 28 aa, Artificial Sequence
  Synthetic polypeptide
SLRKSSCFGRKMDRIREASGLGCNSFRY

SEQ ID NO: 104 - protein, 28 aa, Artificial Sequence
  Synthetic polypeptide
SLRRSSCFGRKMDRIREASGLGCNTFRY

SEQ ID NO: 105 - protein, 28 aa, Artificial Sequence
  Synthetic polypeptide
SLRRSSCFGRKMDRIREASGLGCNSLRY

SEQ ID NO: 106 - protein, 28 aa, Artificial Sequence
  Synthetic polypeptide
SLRRSSCFGRKMDRIREASGLGCNSFKY

SEQ ID NO: 107 - protein, 28 aa, Artificial Sequence
  Synthetic polypeptide
SLRRSSCFGRKMDRIREASGLGCNSFRF

SEQ ID NO: 108 - protein, 32 aa, Artificial Sequence
  Synthetic polypeptide
SAPRSLRRSSCFGRKMDRIREASGLGCNSFRY

SEQ ID NO: 109 - protein, 32 aa, Artificial Sequence
  Synthetic polypeptide
TVPRSLRRSSCFGRKMDRIREASGLGCNSFRY

SEQ ID NO: 110 - protein, 32 aa, Artificial Sequence
  Synthetic polypeptide
TAGRSLRRSSCFGRKMDRIREASGLGCNSFRY

SEQ ID NO: 111 - protein, 32 aa, Artificial Sequence
  Synthetic polypeptide
TAPKSLRRSSCFGRKMDRIREASGLGCNSFRY

SEQ ID NO: 112 - protein, 32 aa, Artificial Sequence
  Synthetic polypeptide
TAPRSLRRSSCFGRKMDRIREASGLGCNTFRY

SEQ ID NO: 113 - protein, 32 aa, Artificial Sequence
  Synthetic polypeptide
TAPRSLRRSSCFGRKMDRIREASGLGCNSLRY

SEQ ID NO: 114 - protein, 32 aa, Artificial Sequence
  Synthetic polypeptide
TAPRSLRRSSCFGRKMDRIREASGLGCNSFKY

SEQ ID NO: 115 - protein, 32 aa, Artificial Sequence
  Synthetic polypeptide
TAPRSLRRSSCFGRKMDRIREASGLGCNSFRF

SEQ ID NO: 116 - protein, 29 aa, Artificial Sequence
  Synthetic polypeptide
TLRRSSCFGLKLDRIREASGLGCKVLRRH

SEQ ID NO: 117 - protein, 29 aa, Artificial Sequence
  Synthetic polypeptide
SIRRSSCFGLKLDRIREASGLGCKVLRRH

SEQ ID NO: 118 - protein, 29 aa, Artificial Sequence
  Synthetic polypeptide
SLKRSSCFGLKLDRIREASGLGCKVLRRH

SEQ ID NO: 119 - protein, 29 aa, Artificial Sequence
  Synthetic polypeptide
SLRKSSCFGLKLDRIREASGLGCKVLRRH

SEQ ID NO: 120 - protein, 29 aa, Artificial Sequence
  Synthetic polypeptide
SLRRSSCFGLKLDRIREASGLGCKVIRRH

SEQ ID NO: 121 - protein, 29 aa, Artificial Sequence
  Synthetic polypeptide
SLRRSSCFGLKLDRIREASGLGCKVLKRH

SEQ ID NO: 122 - protein, 29 aa, Artificial Sequence
  Synthetic polypeptide
SLRRSSCFGLKLDRIREASGLGCKVLRKH

SEQ ID NO: 123 - protein, 29 aa, Artificial Sequence
  Synthetic polypeptide
SLRRSSCFGLKLDRIREASGLGCKVLRRR

SEQ ID NO: 124 - protein, 32 aa, Artificial Sequence
  Synthetic polypeptide
TPKMVQGSGCFGLKLDRIREASGLGCKVLRRH

SEQ ID NO: 125 - protein, 32 aa, Artificial Sequence
  Synthetic polypeptide
SGKMVQGSGCFGLKLDRIREASGLGCKVLRRH

SEQ ID NO: 126 - protein, 32 aa, Artificial Sequence
  Synthetic polypeptide
SPRMVQGSGCFGLKLDRIREASGLGCKVLRRH

SEQ ID NO: 127 - protein, 32 aa, Artificial Sequence
  Synthetic polypeptide
SPKLVQGSGCFGLKLDRIREASGLGCKVLRRH

SEQ ID NO: 128 - protein, 32 aa, Artificial Sequence
  Synthetic polypeptide
SPKMVQGSGCFGLKLDRIREASGLGCKVIRRH

SEQ ID NO: 129 - protein, 32 aa, Artificial Sequence
  Synthetic polypeptide
SPKMVQGSGCFGLKLDRIREASGLGCKVLKRH

SEQ ID NO: 130 - protein, 32 aa, Artificial Sequence
  Synthetic polypeptide
SPKMVQGSGCFGLKLDRIREASGLGCKVLRKH

SEQ ID NO: 131 - protein, 32 aa, Artificial Sequence
  Synthetic polypeptide
SPKMVQGSGCFGLKLDRIREASGLGCKVLRRR

SEQ ID NO: 132 - protein, 38 aa, Artificial Sequence
  Synthetic polypeptide
DVKYDPCFGLKLDRIREASGLGCPSLRDPRPNAPSTSA

SEQ ID NO: 133 - protein, 38 aa, Artificial Sequence
  Synthetic polypeptide
ELKYDPCFGLKLDRIREASGLGCPSLRDPRPNAPSTSA

SEQ ID NO: 134 - protein, 38 aa, Artificial Sequence
  Synthetic polypeptide
EVRYDPCFGLKLDRIREASGLGCPSLRDPRPNAPSTSA

SEQ ID NO: 135 - protein, 38 aa, Artificial Sequence
  Synthetic polypeptide
EVKFDPCFGLKLDRIREASGLGCPSLRDPRPNAPSTSA

SEQ ID NO: 136 - protein, 38 aa, Artificial Sequence
  Synthetic polypeptide
EVKYDPCFGLKLDRIREASGLGCPSLRDPRPNAPTTSA

SEQ ID NO: 137 - protein, 38 aa, Artificial Sequence
  Synthetic polypeptide
EVKYDPCFGLKLDRIREASGLGCPSLRDPRPNAPSSSA

SEQ ID NO: 138 - protein, 38 aa, Artificial Sequence
  Synthetic polypeptide
EVKYDPCFGLKLDRIREASGLGCPSLRDPRPNAPSTTA

SEQ ID NO: 139 - protein, 38 aa, Artificial Sequence
  Synthetic polypeptide
EVKYDPCFGLKLDRIREASGLGCPSLRDPRPNAPSTSV

SEQ ID NO: 140 - protein, 33 aa, Artificial Sequence
  Synthetic polypeptide
SAPRSLRRSSCFGLKLDRIREASGLGCKVLRRH

SEQ ID NO: 141 - protein, 33 aa, Artificial Sequence
  Synthetic polypeptide
TVPRSLRRSSCFGLKLDRIREASGLGCKVLRRH

SEQ ID NO: 142 - protein, 33 aa, Artificial Sequence
  Synthetic polypeptide
TAGRSLRRSSCFGLKLDRIREASGLGCKVLRRH

SEQ ID NO: 143 - protein, 33 aa, Artificial Sequence
  Synthetic polypeptide
TAPKSLRRSSCFGLKLDRIREASGLGCKVLRRH

SEQ ID NO: 144 - protein, 33 aa, Artificial Sequence
  Synthetic polypeptide
TAPRSLRRSSCFGLKLDRIREASGLGCKVIRRH

SEQ ID NO: 145 - protein, 33 aa, Artificial Sequence
  Synthetic polypeptide
TAPRSLRRSSCFGLKLDRIREASGLGCKVLKRH

SEQ ID NO: 146 - protein, 33 aa, Artificial Sequence
  Synthetic polypeptide
TAPRSLRRSSCFGLKLDRIREASGLGCKVLRKH

SEQ ID NO: 147 - protein, 33 aa, Artificial Sequence
  Synthetic polypeptide
TAPRSLRRSSCFGLKLDRIREASGLGCKVLRRR

SEQ ID NO: 148 - protein, 33 aa, Artificial Sequence
  Synthetic polypeptide
KMDRIGLSKGCFGLKLDRIREASGLGCKVLRRH

SEQ ID NO: 149 - protein, 33 aa, Artificial Sequence
  Synthetic polypeptide
RLDRIGLSKGCFGLKLDRIREASGLGCKVLRRH

SEQ ID NO: 150 - protein, 33 aa, Artificial Sequence
  Synthetic polypeptide
RMERIGLSKGCFGLKLDRIREASGLGCKVLRRH

SEQ ID NO: 151 - protein, 33 aa, Artificial Sequence
  Synthetic polypeptide
RMDKIGLSKGCFGLKLDRIREASGLGCKVLRRH

SEQ ID NO: 152 - protein, 33 aa, Artificial Sequence; identical to SEQ ID NO: 151
  Synthetic polypeptide

SEQ ID NO: 153 - protein, 33 aa, Artificial Sequence
  Synthetic polypeptide
RMDRIGLSKGCFGLKLDRIREASGLGCKVLKRH

SEQ ID NO: 154 - protein, 33 aa, Artificial Sequence
  Synthetic polypeptide
RMDRIGLSKGCFGLKLDRIREASGLGCKVLRKH

SEQ ID NO: 155 - protein, 33 aa, Artificial Sequence
  Synthetic polypeptide
RMDRIGLSKGCFGLKLDRIREASGLGCKVLRRR

SEQ ID NO: 156 - protein, 28 aa, Artificial Sequence
  Synthetic polypeptide
PLSKGCFGLKLDRIREASGLGCKVLRRH

SEQ ID NO: 157 - protein, 28 aa, Artificial Sequence
  Synthetic polypeptide
GISKGCFGLKLDRIREASGLGCKVLRRH

SEQ ID NO: 158 - protein, 28 aa, Artificial Sequence
  Synthetic polypeptide
GLTKGCFGLKLDRIREASGLGCKVLRRH

SEQ ID NO: 159 - protein, 28 aa, Artificial Sequence
  Synthetic polypeptide
GLSRGCFGLKLDRIREASGLGCKVLRRH

SEQ ID NO: 160 - protein, 28 aa, Artificial Sequence
  Synthetic polypeptide
GLSKGCFGLKLDRIREASGLGCKVIRRH

SEQ ID NO: 161 - protein, 28 aa, Artificial Sequence
  Synthetic polypeptide
GLSKGCFGLKLDRIREASGLGCKVLKRH

SEQ ID NO: 162 - protein, 28 aa, Artificial Sequence
  Synthetic polypeptide
GLSKGCFGLKLDRIREASGLGCKVLRKH

SEQ ID NO: 163 - protein, 28 aa, Artificial Sequence
  Synthetic polypeptide
GLSKGCFGLKLDRIREASGLGCKVLRRR

SEQ ID NO: 164 - protein, 17 aa, Homo sapiens
CFGGRMDRIGAQSGLGC

SEQ ID NO: 165 - protein, 17 aa, Homo sapiens
CFGRKMDRISSSSGLGC

SEQ ID NO: 166 - protein, 17 aa, Homo sapiens
CFGLKLDRIGSMSGLGC

SEQ ID NO: 167 - protein, 17 aa, Dendroaspis
CFGHKIDRINHVSNLGC

(19) United States
(12) Patent Application Publication
(10) Pub. No.: US 2012/0053123 A1
(43) Pub. Date: Mar. 1, 2012

(54) NATRIURETIC POLYPEPTIDES HAVING MUTATIONS WITHIN THEIR DISULFIDE RINGS

(75) Inventors: John C. Burnett, JR., Rochester, MN (US); Horng H. Chen, Rochester, MN (US)

(73) Assignee: MAYO FOUNDATION FOR MEDICAL EDUCATION AND RESEARCH, Rochester, MN (US)

(21) Appl. No.: 13/266,869

(22) PCT Filed: May 5, 2010
(86) PCT No.: PCT/US10/33700

§ 371 Date: Oct. 28, 2011

Related U.S. Application Data
(60) Provisional application No. 61/175,594, filed on May 5, 2009.

(51) Int. Cl. A61K 38/22; A61P 9/00; A61P 13/12; C07K 14/58; C07H 21/04; C12N 5/10; C12N 15/63; C07K 4/00

(52) U.S. Cl. 514/12.4; 435/320.1; 530/300; 530/324; 536/23.1; 435/325

(57) ABSTRACT

Materials and Methods related to making and using natriuretic polypeptides having a mutation in the ring portion of their structure.

### CROSS-REFERENCE TO RELATED APPLICATIONS

This application claims benefit of priority form U.S. Provisional Application Ser. No. 61/175,594, filed on May 5, 2009.

### TECHNICAL FIELD

This document relates to natriuretic polypeptides. For example, this document provides methods and materials related to making and using natriuretic polypeptides.

### BACKGROUND

Natriuretic polypeptides (NPs) are polypeptides that can cause natriuresis (increased sodium excretion in the urine). Such polypeptides can be produced by brain, heart, kidney, and/or vascular tissue. The NP family in humans includes the cardiac hormones atrial natriuretic peptide (ANP), B-type natriuretic peptide (BNP), C-type natriuretic peptide (CNP), and urodilatin (URO). Natriuretic polypeptides function via well-characterized guanylyl cyclase receptors (i.e., NPR-A for ANP, BNP, and URO; and NPR-B for CNP) and the second messenger cyclic 3′5′ guanosine monophosphate (cGMP) (Kuhn (2003) Circ Res 93:700-709; Tawaragi et al. (1991) Biochem. Biophys. Res. Commun. 175:645-651; and Komatsu et al. (1991) Endocrinol. 129:1104-1106).

### SUMMARY

This document is based in part on the discovery that CNP having mutations within its ring structures can have reduced vascular and hypotensive activities but enhanced renal diuretic activities. For example, CNP having amino acid substitutions at positions 10, 11, and 12 of its ring structure (e.g., having an Arg-Glu-Ala sequence substituted for the amino acid sequence found at positions 10-12 of the native NP ring) can have low or no hypotensive activity relative to a corresponding CNP that does not contain substitutions at positions 10-12 of the ring.

In one aspect, this document features a polypeptide comprising the amino acid sequence set forth in SEQ ID NO:164, SEQ ID NO:165, SEQ ID NO:166, or SEQ ID NO:167, wherein the polypeptide comprises an amino acid substitution at each of positions 10, 11, and 12 of SEQ ID NO:164, SEQ ID NO:165, SEQ ID NO:166, or SEQ ID NO:167, and wherein the polypeptide does not comprise the sequence set forth in SEQ ID NO:5. The amino acids at positions 10, 11, and 12 of SEQ ID NO:164, SEQ ID NO:165, SEQ ID NO:166, or SEQ ID NO:167 can be substituted with arginine, glutamic acid, and alanine, respectively. The polypeptide can have natriuretic activity. The polypeptide can have the sequence set forth in SEQ ID NO:23, SEQ ID NO:25, SEQ ID NO:30, SEQ ID NO:34, SEQ ID NO:36, SEQ ID NO:38, SEQ ID NO:40, SEQ ID NO:43, SEQ ID NO:46, SEQ ID NO:48, or SEQ ID NO:51. The polypeptide can have the sequence set forth in SEQ ID NO:5, 23, 25, 30, 34, 36, 38, 40, 43, 46, 48, or 51, with the proviso that the polypeptide comprises one to three conservative amino acid substitutions, or with the proviso that the polypeptide comprises one to five conservative amino acid substitutions. The polypeptide can be a substantially pure polypeptide.

This document also features an isolated nucleic acid encoding the polypeptide described herein, a vector comprising a nucleic acid encoding the polypeptide, a host cell (e.g., a eukaryotic host cell) comprising a nucleic acid encoding the polypeptide, and a pharmaceutical composition comprising the polypeptide and a pharmaceutically acceptable carrier.

In another aspect, this document features a method for increasing natriuretic activity within a mammal, comprising administering a polypeptide as described herein to the mammal.

In another aspect, this document features a method for treating a mammal having a cardiovascular condition or renal condition, comprising administering to the mammal a polypeptide as described herein, under conditions wherein the severity of a manifestation of the cardiovascular condition or renal condition is reduced.

In yet another aspect, this document features a method for reducing cardiac remodeling in a subject identified as being in need thereof, comprising administering to the subject a pharmaceutical composition as described herein, wherein the composition is administered in an amount effective to alter the level of one or more parameters of cardiac remodeling by at least ten percent as compared to the levels of the one or more parameters prior to administering the composition, and wherein the one or more parameters are selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.

This document also features a method for reducing restenosis in a subject identified as being in need thereof, comprising administering to the subject a restenosis-reducing amount of a pharmaceutical composition as described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the ABC-NP1 polypeptide. The amino acid sequence of ABC-NP1 (RMDRIGLSKGCFGLKLDRIGSMSGLGCKVLRRH; SEQ ID NO:1) includes five amino acids (residues 11-15) from ANP (RMDRI; SEQ ID NO:2), followed by the amino acid sequence of CNP (GLSKGCFGLKLDRIGSMSGLGC; SEQ ID NO:3), and a six amino acids from the C-terminus of BNP (KVLRRH; SEQ ID NO:4).

FIG. 2 is a diagram of the ABC-NP polypeptide. The amino acid sequence of ABC-NP (RMDRIGLSKGCFGLKLDRIREASGLGCKVLRRH; SEQ ID NO:5) includes five amino acids (residues 11-15) from ANP (RMDRI; SEQ ID NO:2), a mutated CNP sequence in which GSM₁₅₋₁₇ of the native CNP sequence (corresponding to amino acids 10-12 of the CNP ring structure) were changed to REA (GLSKGCFGLKLDRIREASGLGC; SEQ ID NO:6), and a six amino acids from the C-terminus of BNP (KVLRRH; SEQ ID NO:4).

FIG. 3 is a graph plotting urine flow (UV) in normal dogs treated with an intravenous (i.v.) bolus of ABC-NP or ABC-NP1, as indicated. *p<0.05.

FIG. 4 is a graph plotting urinary sodium excretion (UNaV) in normal dogs treated with an i.v. bolus of ABC-NP or ABC-NP1, as indicated. *p<0.05.

FIG. 5 is a graph plotting mean arterial blood pressure (MAP) in normal dogs treated with an i.v. bolus of ABC-NP or ABC-NP1, as indicated. *p<0.05.

FIG. 6 is a graph plotting right atrial pressure (RAP) in normal dogs treated with an i.v. bolus of ABC-NP or ABC-NP1, as indicated. *p<0.05.

FIG. 7 is a graph plotting pulmonary capillary wedge pressure (PCWP) in normal dogs treated with an i.v. bolus of ABC-NP or ABC-NP1, as indicated. *p<0.05.

FIG. 8 is a graph plotting cGMP generation in cultured human cardiac fibroblasts (HCF) treated with ABC-NP or ABC-NP1, as indicated.

FIG. 9 is a graph plotting cGMP generation in cultured human aortic endothelial cells (HAEC) treated with ABC-NP or ABC-NP1, as indicated.

FIG. 10 is a graph plotting cGMP generation in isolated canine glomeruli treated with ABC-NP or ABC-NP1, as indicated.

FIG. 11 is a schematic of the protocol used in the study discussed in Example 2 herein.

FIG. 12 is a graph plotting MAP in dogs having rapid ventricular pacing-induced overt heart failure (HF) with cardiorenal dysfunction, at baseline (BL) and after treatment with 2, 10, and 100 pmol/kg/min of ABC-NP or ABC-NP1. *p<0.05

FIG. 13 is a graph plotting RAP in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 14 is a graph plotting PCWP in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 15 is a graph plotting UNaV in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 16 is a graph plotting UV in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 17 is a graph plotting glomerular filtration rate (GFR) in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 18 is a graph plotting renal blood flow in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 19 is a graph plotting urinary cGMP excretion in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 20 is a graph plotting plasma cGMP levels in dogs having rapid ventricular pacing-induced overt HF with cardiorenal dysfunction, at baseline (BL) and after treatment with 2, 10, and 100 pmol/kg/min of ABC-NP or ABC-NP1. *p<0.05

FIG. 21 is a graph plotting levels of proliferation of vascular smooth muscle cells after treatment with 10⁻⁵ M, 10⁻⁶ M, 10⁻⁷ M, or 10⁻⁸ M ANP, BNP, CNP, ABC-NP, or ABC-NP1, measured by assessing bromodeoxyuridine (BrdU) levels.

### DETAILED DESCRIPTION

### Natriuretic Compounds

This document provides natriuretic compounds (e.g., polypeptides) and compositions that can be used to increase natriuretic activity in a subject in need thereof. For example, isolated NPs can increase plasma cGMP levels, urinary cGMP excretion, net renal cGMP generation, urine flow, urinary sodium excretion, urinary potassium excretion, hematocrit, plasma BNP immunoreactivity, renal blood flow, and/or plasma ANP immunoreactivity, and decrease renal vascular resistance, proximal and distal fractional reabsorption of sodium, mean arterial pressure, pulmonary capillary wedge pressure, right atrial pressure, pulmonary arterial pressure, plasma renin activity, plasma angiotensin II levels, plasma aldosterone levels, renal perfusion pressure, and/or systemic vascular resistance. NPs also may be useful to reduce or prevent restenosis that can occur, for example, after vascular surgery, cardiac surgery, interventional radiology, or interventional cardiology following angioplasty or stent placement. Further, NPs may be useful to prevent, reduce, and/or inhibit cardiac remodeling and prevent, reduce, and/or w inhibit ischemic injury after myocardial infarction (MI).

As used herein, the term “natriuretic polypeptide” or “NP” includes native (naturally occurring, wild type) NPs (e.g., human ANP, BNP, CNP, and URO, as well as Dendroaspis natriuretic peptide (DNP)), one or more portions of a native NP, variants of a native NP, or chimeras of native NPs, portions of native NPs, or variants of native NPs or portions of native NPs. In some embodiments, a NP includes only portions of the mature form of a native NP. Chimeric NPs containing amino acid sequences from two or more of human CNP, BNP, ANP, URO, and/or Dendroaspis DNP can be particularly useful, although other NPs are contemplated herein.

An “isolated” polypeptide is a polypeptide that (1) is not associated with proteins found in nature, (2) is free of other proteins from the same source (e.g., free of human proteins), (3) is expressed by a cell from a different species, or (4) does not occur in nature. An isolated polypeptide can be, for example, encoded by DNA or RNA, including synthetic DNA or RNA, or some combination thereof.

Amino acid sequences for endogenous human mature NPs include the following:

(SEQ ID NO: 7)
ANP: SLRRSSCFGGRMDRI [symbol] SGLGCNSFRY
(SEQ ID NO: 8)
BNP: SPKMVQGSGCFGRKMDRI [symbol] SGLGCKVLRRH
(SEQ ID NO: 3)
CNP: GLSKGCFGLKLDRI [symbol] SGLGC
(SEQ ID NO: 9)
URO: TAPRSLRRSSCFGGRMDRI [symbol] SGLGCNSFRY

In addition, the native Dendroaspis amino acid sequence for DNP is

(SEQ ID NO: 10)
EVKYDPCFGHKIDRI [symbol] SNLGCPSLRDPRPNAPSTSA.

Each of these native mature NPs includes a 17-amino acid ring structure with a cysteine bond between the cysteine residues at positions 1 and 17 (underlined in the above sequences) of the ring. The ring structure of an NP can include one or more (e.g., one, two, three, four, five, six, or more than six) amino acid substitutions. In some embodiments, a NP can have a ring structure in which the amino acids at positions 10, 11, and 12 (italicized and bolded in the above sequences) are mutated from the native sequence. For example, NPs with the following sequences are provided herein:

(SEQ ID NO: 11)
SLRRSSCFGGRMDRI [symbol] SGLGCNSFRY
(SEQ ID NO: 12)
SLRRSSCFGGRMDRI [symbol] SGLGCNSFRY
(SEQ ID NO: 13)
SPKMVQGSGCFGRKMDRI [symbol] SGLGCKVLRRH
(SEQ ID NO: 14)
SPKMVQGSGCFGRKMDRI [symbol] SGLGCKVLRRH
(SEQ ID NO: 15)
GLSKGCFGLKLDRI [symbol] SGLGC
(SEQ ID NO: 6)
GLSKGCFGLKLDRI [symbol] SGLGC
(SEQ ID NO: 16)
TAPRSLRRSSCFGGRMDRI [symbol] SGLGCNSFRY
(SEQ ID NO: 17)
TAPRSLRRSSCFGGRMDRI [symbol] SGLGCNSFRY
(SEQ ID NO: 18)
EVKYDPCFGHKIDRI [symbol] SNLGCPSLRDPRPNAPSTSA
(SEQ ID NO: 19)
EVKYDPCFGHKIDRI [symbol] SNLGCPSLRDPRPNAPSTSA

Mutated amino acids in SEQ ID NOS:11-19 are italicized and bolded, and X can be any amino acid other than the native amino acid at that position. While substitution of the three native amino acids in positions 10-12 with the sequence REA can be particularly useful, other substitutions are contemplated herein.

This document also provides chimeric NPs, which can include amino acid sequences from two or more individual NPs. In some embodiments, for example, a chimeric polypeptide can include amino acid sequences from ANP and CNP; BNP and CNP; ANP, BNP, and CNP; CNP and URO; CNP and DNP; or CNP, URO, and BNP. Chimeric NPs typically include a ring structure and cysteine bond from one NP (e.g., the ring structure and cysteine bond of ANP, BNP, CNP, DNP, or URO) in combination with one or more amino acid segments from another NP. The ring structure can include one or more (e.g., one, two, three, four, five, six, or more than six) amino acid substitutions. For example, a chimeric NP can have a ring structure in which the amino acids at positions 10, 11, and 12 are mutated from the native sequence of the ring structure. The chimeric NPs described herein are non-limiting examples of polypeptides that can be useful to increase diuresis and/or natriuresis, for example.

In some embodiments, a chimeric NP can include the N-terminal 26 amino acids of human BNP with substitutions at amino acids 10-12 of the ring (SPKMVQGSGCFGRKMDRIXXXSGLGC; SEQ ID NO:20), where X is any amino acid other than the native amino acid at that position, and the C-terminal 15 amino acids of DNP (PSLRDPRPNAPSTSA; SEQ ID NO:21), and can have the amino acid sequence SPKMVQGSGCFGRKMDRIXXXSGLG CPSLRDPRPNAPSTSA (SEQ ID NO:22). In some cases, a chimeric NP can have the amino acid sequence

(SEQ ID NO: 23)
SPKMVQGSGCFGRKMDRI [symbol] SGLGCPSLRDPRPNAPSTSA.

In some embodiments, a chimeric NP can include the amino acid sequence of human CNP with substitutions at amino acids 10-12 of the ring (GLSKGCFGL KLDRIXXXSGLGC; SEQ ID NO:15), where X is any amino acid other than the native amino acid at that position, and the C-terminal 15 amino acids of DNP (PSLRDPRPNAPSTSA; SEQ ID NO:21), and can have the amino acid sequence GLSKGCFGLKLDRIXXXSGLGCPSLRDPRPNAPSTSA (SEQ ID NO:24). In some cases, a chimeric NP can have the amino acid sequence

(SEQ ID NO: 25)
GLSKGCFGLKLDRI [symbol] SGLGCPSLRDPRPNAPSTSA.

In some embodiments, a chimeric NP can include the N-terminal ten amino acids of human URO (TAPRSLRRSS; SEQ ID NO:26), the 17 amino acid ring structure and disulfide bond of human CNP with substitutions at amino acids 10-12 of the ring (CFGLKLDRIXXXSGLGC; SEQ ID NO:27), where X is any amino acid other than the native amino acid at that position, and the C-terminal five amino acids of human URO (NSFRY; SEQ ID NO:28), and can have the amino acid sequence TAPRSLRRSSCFGLKLDRLXXXSGLGCNSFRY (SEQ ID NO:29). In some cases, a chimeric NP can have the amino acid sequence

| | |
|---|---|
| TAPRSLRRSSCFGLKLDRI [symbol] SGLGCNSFRY. | (SEQ ID NO: 30) |

In some embodiments, a chimeric NP can include the N-terminal six amino acids of human ANP (SLRRSS; SEQ ID NO:31), the 17 amino acid ring structure and disulfide bond of human BNP with substitutions at amino acids 10-12 of the ring structure (CFGRKMDRIXXXSGLGC; SEQ ID NO:32), where X is any amino acid other than the native amino acid at that position, and the C-terminal five amino acids of human ANP (NSFRY; SEQ ID NO:28), and can have the amino acid sequence SLRRSSCFGRKMDRIXXXSGLGCNSFRY (SEQ ID NO:33). In some cases, a chimeric NP can have the amino acid sequence

| | |
|---|---|
| SLRRSSCFGRKMDRI [symbol] SGLGCNSFRY. | (SEQ ID NO: 34) |

In some embodiments, a chimeric NP can include the N-terminal 10 amino acids of human URO (TAPRSLRRSS; SEQ ID NO:26), the 17 amino acid ring structure and disulfide bond of human BNP with substitutions at amino acids 10-12 of the ring structure (CFGRKMDRIXXXSGLGC; SEQ ID NO:32), where X is any amino acid other than the native amino acid at that position, and the C-terminal 5 amino acids of human ANP (NSFRY; SEQ ID NO:28), and can have the amino acid sequence TAPRSLRRSSCFGRKMDRIXXXSGLGCNSFRY (SEQ ID NO:35). In some cases, a chimeric NP can have the amino acid sequence

| | |
|---|---|
| TAPRSLRRSSCFGRKMDRI [symbol] SGLGCNSFRY. | (SEQ ID NO: 36) |

In some embodiments, a chimeric NP can include the N-terminal 6 amino acids of human ANP (SLRRSS; SEQ ID NO:31), the 17 amino acid ring structure and disulfide bond of human CNP with substitutions at amino acids 10-12 of the ring structure (CFGLKLDRIXXXSGLGC; SEQ ID NO:27), where X is any amino acid other than the native amino acid at that position, and the C-terminal 5 amino acids of human ANP (NSFRY; SEQ ID NO:28), and can have the amino acid sequence SLRRSSCFGLKLDRIXXXSGLGCNSFRY (SEQ ID NO:37). In some cases, a chimeric NP can have the amino acid sequence SLRRSSCFGLKLDRIREASGLGCNSFRY (SEQ ID NO:38).

As another example, in some embodiments, a chimeric NP can include the N-terminal six amino acids of human ANP (SLRRSS; SEQ ID NO:31), the 17 amino acid ring structure and disulfide bond of human CNP with substitutions at amino acids 10-12 of the ring structure (CFGLKLDRIXXXSGLGC; SEQ ID NO:27), where X is any amino acid other than the native amino acid at that position, and the C-terminal six amino acids of human BNP (KVLRRH; SEQ ID NO:4), and can have the amino acid sequence SLRRSSCFGLKLDRIXXXSGLGCKVLRRH (SEQ ID NO:39). In some cases, a chimeric NP can have the amino acid sequence

| | |
|---|---|
| SLRRSSCFGLKLDRI [symbol] SGLGCKVLRRH. | (SEQ ID NO: 40) |

In some embodiments, a chimeric NP can include the N-terminal nine amino acids of human BNP (SPKMVQGSG; SEQ ID NO:41), the 17 amino acid ring structure and disulfide bond of human CNP with substitutions at amino acids 10-12 of the ring structure (CFGLKLDRIXXXSGLGC; SEQ ID NO:27), where X is any amino acid other than the native amino acid at that position, and the C-terminal six amino acids of human BNP (KVLRRH; SEQ ID NO:4), and can have the amino acid sequence SPKMVQGSGCFGLKLDRIXXXSGLGCKVLRRH (SEQ ID NO:42). In some cases, a chimeric NP can have the amino acid sequence

| | |
|---|---|
| SPKMVQGSGCFGLKLDRI [symbol] SGLGCKVLRRH. | (SEQ ID NO: 43) |

In some embodiments, a chimeric NP can include the N-terminal six amino acids of DNP (EVKYDP; SEQ ID NO:44), the 17 amino acid ring structure and disulfide bond of human CNP with substitutions at amino acids 10-12 of the ring structure (CFGLKLDRIXXXSGLGC; SEQ ID NO:27), where X is any amino acid other than the native amino acid at that position, and the C-terminal 15 amino acids of DNP (PSLRDPRPNAPSTSA; SEQ ID NO:21), and can have the amino acid sequence EVKYDPCFGLKLDRIXXXSGLGCPSLRDPRPNAPSTSA (SEQ ID NO:45). In some cases, a chimeric NP can have the amino acid sequence

(SEQ ID NO: 46)
EVKYDPCFGLKLDRI [symbol] SGLGCPSLRDPRPNAPSTSA.

In some embodiments, a chimeric NP can include the N-terminal 10 amino acids of human URO (TAPRSLRRSS; SEQ ID NO:26), the 17 amino acid ring structure and disulfide bond of human CNP with substitutions at amino acids 10-12 of the ring structure (CFGLKLDRIXXXSGLGC; SEQ ID NO:27), where X is any amino acid other than the native amino acid at that position, and the C-terminal six amino acids of human BNP (KVLRRH; SEQ ID NO:4), and can have the amino acid sequence TAPRSLRRSSCFGLKLDRLXXXSGLGCKVLRRH (SEQ ID NO:47). In some cases, a chimeric NP can have the amino acid sequence

| | |
|---|---|
| TAPRSLRRSSCFGLKLDRI [symbol] SGLGCKVLRRH. | (SEQ ID NO: 48) |

In some embodiments, a chimeric NP can include amino acids 11 to 15 of human ANP (RMDRI; SEQ ID NO:2) at its amino terminus, followed by the amino acid sequence of human CNP with substitutions at amino acids 10-12 of the ring structure (GLSKGCFGLKLDRIXXXSGLGC; SEQ ID NO:15), where X is any amino acid other than the native amino acid at that position, and the C-terminal six amino acids of human BNP (KVLRRH; SEQ ID NO:4), and can have the amino acid sequence RMDRIGLSKGCFGLKLDRLXXXSGLGCKVLRRH (SEQ ID NO:49). In some cases, a chimeric NP can have the amino acid sequence

| | |
|---|---|
| RMDRIGLSKGCFGLKLDRI [symbol] SGLGCKVLRRH. | (SEQ ID NO: 5) |

In some embodiments, a chimeric NP can include the amino acid sequence of human CNP with substitutions at amino acids 10-12 of the ring structure (GLSKGCFGLKLDRIXXXSGLGC; SEQ ID NO:15), where X is any amino acid other than the native amino acid at that position, followed by the C-terminal six amino acids of human BNP (KVLRRH; SEQ ID NO:4), and can have the amino acid sequence GLSKGCFGLKLDRIXXXSGLGCKVLRRH (SEQ ID NO:50). In some cases, a chimeric NP can have the amino acid sequence

| | |
|---|---|
| GLSKGCFGLKLDRI [symbol] SGLGCKVLRRH. | (SEQ ID NO: 51) |

In some embodiments, a chimeric NP can include a variant (e.g., a substitution, addition, or deletion) at one or more positions (e.g., one, two, three, four, five, six, seven, eight, nine, or ten positions) with respect to any of SEQ ID NOS:1 to 51, in addition to having substitutions at amino acids 10-12 of the ring structure.

NPs having one or more amino acid substitutions relative to a native NP amino acid sequence (also referred to herein as “variant” NPs) can be prepared and modified as described herein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Non-limiting examples of variant chimeric NPs include the following:

(SEQ ID NO: 52)
TLRRSSCFGGRMDRIREASGLGCNSFRY
(SEQ ID NO: 53)
SIRRSSCFGGRMDRIREASGLGCNSFRY
(SEQ ID NO: 54)
SLKRSSCFGGRMDRIREASGLGCNSFRY
(SEQ ID NO: 55)
SLRKSSCFGGRMDRIREASGLGCNSFRY
(SEQ ID NO: 56)
SLRRSSCFGGRMDRIREASGLGCNTFRY
(SEQ ID NO: 57)
SLRRSSCFGGRMDRIREASGLGCNSLRY
(SEQ ID NO: 58)
SLRRSSCFGGRMDRIREASGLGCNSFKY
(SEQ ID NO: 59)
SLRRSSCFGGRMDRIREASGLGCNSFRF
(SEQ ID NO: 60)
TPKMVQGSGCFGRKMDRIREASGLGCKVLRRH
(SEQ ID NO: 61)
SGKMVQGSGCFGRKMDRIREASGLGCKVLRRH
(SEQ ID NO: 62)
SPRMVQGSGCFGRKMDRIREASGLGCKVLRRH
(SEQ ID NO: 63)
SPKLVQGSGCFGRKMDRIREASGLGCKVLRRH
(SEQ ID NO: 64)
SPKMVQGSGCFGRKMDRIREASGLGCKVIRRH
(SEQ ID NO: 65)
SPKMVQGSGCFGRKMDRIREASGLGCKVLKRH
(SEQ ID NO: 66)
SPKMVQGSGCFGRKMDRIREASGLGCKVLRKH
(SEQ ID NO: 67)
SPKMVQGSGCFGRKMDRIREASGLGCKVLRRR
(SEQ ID NO: 68)
PLSKGCFGLKLDRIREASGLGC
(SEQ ID NO: 69)
GISKGCFGLKLDRIREASGLGC
(SEQ ID NO: 70)
GLTKGCFGLKLDRIREASGLGC
(SEQ ID NO: 71)
GLSRGCFGLKLDRIREASGLGC
(SEQ ID NO: 72)
GLSKGCFGLKLDRIREASPLGC
(SEQ ID NO: 73)
GLSKGCFGLKLDRIREASGIGC
(SEQ ID NO: 74)
GLSKGCFGLKLDRIREASGLPC
(SEQ ID NO: 75)
GLSKGCFGLKLDRIREASGLGS
(SEQ ID NO: 76)
TPKMVQGSGCFGRKMDRIREASGLGCPSLRDPRPNAPSTSA
(SEQ ID NO: 77)
SGKMVQGSGCFGRKMDRIREASGLGCPSLRDPRPNAPSTSA
(SEQ ID NO: 78)
SPRMVQGSGCFGRKMDRIREASGLGCPSLRDPRPNAPSTSA
(SEQ ID NO: 79)
SPKLVQGSGCFGRKMDRIREASGLGCPSLRDPRPNAPSTSA
(SEQ ID NO: 80)
SPKMVQGSGCFGRKMDRIREASGLGCPSLRDPRPNAPTTSA
(SEQ ID NO: 81)
SPKMVQGSGCFGRKMDRIREASGLGCPSLRDPRPNAPSSSA
(SEQ ID NO: 82)
SPKMVQGSGCFGRKMDRIREASGLGCPSLRDPRPNAPSTTA
(SEQ ID NO: 83)
SPKMVQGSGCFGRKMDRIREASGLGCPSLRDPRPNAPSTSV
(SEQ ID NO: 84)
PLSKGCFGLKLDRIREASGLGCPSLRDPRPNAPSTSA
(SEQ ID NO: 85)
GISKGCFGLKLDRIREASGLGCPSLRDPRPNAPSTSA
(SEQ ID NO: 86)
GLTKGCFGLKLDRIREASGLGCPSLRDPRPNAPSTSA
(SEQ ID NO: 87)
GLSRGCFGLKLDRIREASGLGCPSLRDPRPNAPSTSA
(SEQ ID NO: 88)
GLSKGCFGLKLDRIREASGLGCPSLRDPRPNAPTTSA
(SEQ ID NO: 89)
GLSKGCFGLKLDRIREASGLGCPSLRDPRPNAPSSSA
(SEQ ID NO: 90)
GLSKGCFGLKLDRIREASGLGCPSLRDPRPNAPSTTA
(SEQ ID NO: 91)
GLSKGCFGLKLDRIREASGLGCPSLRDPRPNAPSTSV
(SEQ ID NO: 92)
SAPRSLRRSSCFGLKLDRIREASGLGCNSFRY
(SEQ ID NO: 93)
TVPRSLRRSSCFGLKLDRIREASGLGCNSFRY
(SEQ ID NO: 94)
TAGRSLRRSSCFGLKLDRIREASGLGCNSFRY
(SEQ ID NO: 95)
TAPKSLRRSSCFGLKLDRIREASGLGCNSFRY
(SEQ ID NO: 96)
TAPRSLRRSSCFGLKLDRIREASGLGCNTFRY
(SEQ ID NO: 97)
TAPRSLRRSSCFGLKLDRIREASGLGCNSLRY
(SEQ ID NO: 98)
TAPRSLRRSSCFGLKLDRIREASGLGCNSFKY
(SEQ ID NO: 99)
TAPRSLRRSSCFGLKLDRIREASGLGCNSFRF
(SEQ ID NO: 100)
TLRRSSCFGRKMDRIREASGLGCNSFRY
(SEQ ID NO: 101)
SIRRSSCFGRKMDRIREASGLGCNSFRY
(SEQ ID NO: 102)
SLKRSSCFGRKMDRIREASGLGCNSFRY
(SEQ ID NO: 103)
SLRKSSCFGRKMDRIREASGLGCNSFRY
(SEQ ID NO: 104)
SLRRSSCFGRKMDRIREASGLGCNTFRY
(SEQ ID NO: 105)
SLRRSSCFGRKMDRIREASGLGCNSLRY
(SEQ ID NO: 106)
SLRRSSCFGRKMDRIREASGLGCNSFKY
(SEQ ID NO: 107)
SLRRSSCFGRKMDRIREASGLGCNSFRF
(SEQ ID NO: 108)
SAPRSLRRSSCFGRKMDRIREASGLGCNSFRY
(SEQ ID NO: 109)
TVPRSLRRSSCFGRKMDRIREASGLGCNSFRY
(SEQ ID NO: 110)
TAGRSLRRSSCFGRKMDRIREASGLGCNSFRY
(SEQ ID NO: 111)
TAPKSLRRSSCFGRKMDRIREASGLGCNSFRY
(SEQ ID NO: 112)
TAPRSLRRSSCFGRKMDRIREASGLGCNTFRY
(SEQ ID NO: 113)
TAPRSLRRSSCFGRKMDRIREASGLGCNSLRY
(SEQ ID NO: 114)
TAPRSLRRSSCFGRKMDRIREASGLGCNSFKY
(SEQ ID NO: 115)
TAPRSLRRSSCFGRKMDRIREASGLGCNSFRF
(SEQ ID NO: 116)
TLRRSSCFGLKLDRIREASGLGCKVLRRH
(SEQ ID NO: 117)
SIRRSSCFGLKLDRIREASGLGCKVLRRH
(SEQ ID NO: 118)
SLKRSSCFGLKLDRIREASGLGCKVLRRH
(SEQ ID NO: 119)
SLRKSSCFGLKLDRIREASGLGCKVLRRH
(SEQ ID NO: 120)
SLRRSSCFGLKLDRIREASGLGCKVIRRH
(SEQ ID NO: 121)
SLRRSSCFGLKLDRIREASGLGCKVLKRH
(SEQ ID NO: 122)
SLRRSSCFGLKLDRIREASGLGCKVLRKH
(SEQ ID NO: 123)
SLRRSSCFGLKLDRIREASGLGCKVLRRR
(SEQ ID NO: 124)
TPKMVQGSGCFGLKLDRIREASGLGCKVLRRH
(SEQ ID NO: 125)
SGKMVQGSGCFGLKLDRIREASGLGCKVLRRH
(SEQ ID NO: 126)
SPRMVQGSGCFGLKLDRIREASGLGCKVLRRH
(SEQ ID NO: 127)
SPKLVQGSGCFGLKLDRIREASGLGCKVLRRH
(SEQ ID NO: 128)
SPKMVQGSGCFGLKLDRIREASGLGCKVIRRH
(SEQ ID NO: 129)
SPKMVQGSGCFGLKLDRIREASGLGCKVLKRH
(SEQ ID NO: 130)
SPKMVQGSGCFGLKLDRIREASGLGCKVLRKH
(SEQ ID NO: 131)
SPKMVQGSGCFGLKLDRIREASGLGCKVLRRR
(SEQ ID NO: 132)
DVKYDPCFGLKLDRIREASGLGCPSLRDPRPNAPSTSA
(SEQ ID NO: 133)
ELKYDPCFGLKLDRIREASGLGCPSLRDPRPNAPSTSA
(SEQ ID NO: 134)
EVRYDPCFGLKLDRIREASGLGCPSLRDPRPNAPSTSA
(SEQ ID NO: 135)
EVKFDPCFGLKLDRIREASGLGCPSLRDPRPNAPSTSA
(SEQ ID NO: 136)
EVKYDPCFGLKLDRIREASGLGCPSLRDPRPNAPTTSA
(SEQ ID NO: 137)
EVKYDPCFGLKLDRIREASGLGCPSLRDPRPNAPSSSA
(SEQ ID NO: 138)
EVKYDPCFGLKLDRIREASGLGCPSLRDPRPNAPSTTA
(SEQ ID NO: 139)
EVKYDPCFGLKLDRIREASGLGCPSLRDPRPNAPSTSV
(SEQ ID NO: 140)
SAPRSLRRSSCFGLKLDRIREASGLGCKVLRRH
(SEQ ID NO: 141)
TVPRSLRRSSCFGLKLDRIREASGLGCKVLRRH
(SEQ ID NO: 142)
TAGRSLRRSSCFGLKLDRIREASGLGCKVLRRH
(SEQ ID NO: 143)
TAPKSLRRSSCFGLKLDRIREASGLGCKVLRRH
(SEQ ID NO: 144)
TAPRSLRRSSCFGLKLDRIREASGLGCKVIRRH
(SEQ ID NO: 145)
TAPRSLRRSSCFGLKLDRIREASGLGCKVLKRH
(SEQ ID NO: 146)
TAPRSLRRSSCFGLKLDRIREASGLGCKVLRKH
(SEQ ID NO: 147)
TAPRSLRRSSCFGLKLDRIREASGLGCKVLRRR
(SEQ ID NO: 148)
KMDRIGLSKGCFGLKLDRIREASGLGCKVLRRH
(SEQ ID NO: 149)
RLDRIGLSKGCFGLKLDRIREASGLGCKVLRRH
(SEQ ID NO: 150)
RMERIGLSKGCFGLKLDRIREASGLGCKVLRRH
(SEQ ID NO: 151)
RMDKIGLSKGCFGLKLDRIREASGLGCKVLRRH
(SEQ ID NO: 152)
RMDRIGLSKGCFGLKLDRIREASGLGCKVIRRH
(SEQ ID NO: 153)
RMDRIGLSKGCFGLKLDRIREASGLGCKVLKRH
(SEQ ID NO: 154)
RMDRIGLSKGCFGLKLDRIREASGLGCKVLRKH
(SEQ ID NO: 155)
RMDRIGLSKGCFGLKLDRIREASGLGCKVLRRR
(SEQ ID NO: 156)
PLSKGCFGLKLDRIREASGLGCKVLRRH
(SEQ ID NO: 157)
GISKGCFGLKLDRIREASGLGCKVLRRH
(SEQ ID NO: 158)
GLTKGCFGLKLDRIREASGLGCKVLRRH
(SEQ ID NO: 159)
GLSRGCFGLKLDRIREASGLGCKVLRRH
(SEQ ID NO: 160)
GLSKGCFGLKLDRIREASGLGCKVIRRH
(SEQ ID NO: 161)
GLSKGCFGLKLDRIREASGLGCKVLKRH
(SEQ ID NO: 162)
GLSKGCFGLKLDRIREASGLGCKVLRKH
(SEQ ID NO: 163)
GLSKGCFGLKLDRIREASGLGCKVLRRR

Further examples of conservative substitutions that can be made at any position within the polypeptides provided herein are set forth in Table 1.

TABLE 1
Examples of conservative amino acid substitutions

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, a NP can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the peptide variant using, for example, methods disclosed herein.

A polypeptide provided herein can have any sequence and can have any length. For example, a polypeptide can include the sequences set forth in SEQ ID NO:5, 23, 25, 30, 34, 36, 38, 40, 43, 46, 48, or 51. In some embodiments, a polypeptide provided herein can be between 17 and 45 (e.g., between 18 and 40, between 22 and 44, between 25 and 45, between 26 and 44, between 27 and 43, between 28 and 42, between 29 and 41, between 30 and 40, between 31 and 39, between 23 and 35, between 25 and 30, or between 30 and 35) amino acid residues in length. It will be appreciated that a polypeptide with a length of 17 or 45 amino acid residues is a polypeptide with a length between 17 and 45 amino acid residues.

In some embodiments, a polypeptide can contain the ring structure of human ANP or human URO (CFGGRMDRIGAQSGLGC; SEQ ID NO:164), the ring structure of human BNP (CFGRKMDRISSSSGLGC; SEQ ID NO:165), the ring structure of human CNP (CFGLKLDRIGSMSGLGC; SEQ ID NO:166), or the ring structure of Dendroapsis DNP (CFGHKIDRINHVSNLGC, SEQ ID NO:167), wherein the amino acids at positions 10, 11, and 12 of SEQ ID NO:164, 165, 166, or 167 are replaced by other amino acids (such as, without limitation, Arg-Glu-Ala).

In some cases, a polypeptide provided herein can contain an amino acid sequence that aligns to (a) the sequence set forth in SEQ ID NO:5 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:5, with the exception that the first arginine residue or the last histidine residue of SEQ ID NO:5 is deleted or replaced with a different amino acid residue.

In some cases, a polypeptide provided herein can contain an amino acid sequence that aligns to (a) the sequence set forth in SEQ ID NO:23 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:23, with the exception that the first serine residue or the last alanine residue of SEQ ID NO:23 is deleted or replaced with a different amino acid residue.

In some cases, a polypeptide provided herein can contain an amino acid sequence that aligns to (a) the sequence set forth in SEQ ID NO:25 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:25, with the exception that the first glycine residue or the last alanine residue of SEQ ID NO:25 is deleted or replaced with a different amino acid residue.

In some cases, a polypeptide provided herein can contain an amino acid sequence that aligns to (a) the sequence set forth in SEQ ID NO:30 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:30, with the exception that the first threonine residue or the last tyrosine residue of SEQ ID NO:30 is deleted or replaced with a different amino acid residue.

In some cases, a polypeptide provided herein can contain an amino acid sequence that aligns to (a) the sequence set forth in SEQ ID NO:34 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:34, with the exception that the first serine residue or the last tyrosine residue of SEQ ID NO:34 is deleted or replaced with a different amino acid residue.

In some cases, a polypeptide provided herein can contain an amino acid sequence that aligns to (a) the sequence set forth in SEQ ID NO:36 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:36, with the exception that the first threonine residue or the last tyrosine residue of SEQ ID NO:36 is deleted or replaced with a different amino acid residue.

In some cases, a polypeptide provided herein can contain an amino acid sequence that aligns to (a) the sequence set forth in SEQ ID NO:38 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:38, with the exception that the first serine residue or the last tyrosine residue of SEQ ID NO:38 is deleted or replaced with a different amino acid residue.

In some cases, a polypeptide provided herein can contain an amino acid sequence that aligns to (a) the sequence set forth in SEQ ID NO:40 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:40, with the exception that the first serine residue or the last histidine residue of SEQ ID NO:40 is deleted or replaced with a different amino acid residue.

In some cases, a polypeptide provided herein can contain an amino acid sequence that aligns to (a) the sequence set forth in SEQ ID NO:43 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:43, with the exception that the first serine residue or the last histidine residue of SEQ ID NO:43 is deleted or replaced with a different amino acid residue.

In some cases, a polypeptide provided herein can contain an amino acid sequence that aligns to (a) the sequence set forth in SEQ ID NO:46 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:46, with the exception that the first glutamic acid residue or the last alanine residue of SEQ ID NO:46 is deleted or replaced with a different amino acid residue.

In some cases, a polypeptide provided herein can contain an amino acid sequence that aligns to (a) the sequence set forth in SEQ ID NO:48 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:48, with the exception that the first threonine residue or the last histidine residue of SEQ ID NO:48 is deleted or replaced with a different amino acid residue.

In some cases, a polypeptide provided herein can contain an amino acid sequence that aligns to (a) the sequence set forth in SEQ ID NO:51 with ten or less (e.g., ten or less, nine or less, eight or less, seven or less, six or less, five or less, four or less, three or less, two or less, one, or zero) amino acid additions, deletions, substitutions, or combinations thereof. For example, a polypeptide provided herein can contain the sequence set forth in SEQ ID NO:51, with the exception that the first glycine residue or the last histidine residue of SEQ ID NO:51 is deleted or replaced with a different amino acid residue.

Variant NPs having conservative and/or non-conservative substitutions (e.g., with respect to any of SEQ ID NOS:1 to 51), as well as fragments of any of SEQ ID NOS:1 to 51, fragments of variants of any of SEQ ID NOS:1 to 51, and polypeptides comprising any of SEQ ID NOS:1 to 51, variants or fragments of any of SEQ ID NOS:1 to 51, or fragments of variants of any of SEQ ID NOS:1 to 51, can be screened for biological activity using any of a number of assays, including those described herein. For example, the activity of a NP as described herein can be evaluated in vitro by testing its effect on cGMP production in cultured cells (e.g., cultured cardiac fibroblasts, aortic endothelial cells, or glomerular cells, as described in the Example herein). Cells can be exposed to a NP (e.g., 10⁻⁹ to 10⁻⁴ M NP), and samples can be assayed to evaluate the NP effects on cGMP generation. cGMP generation can be detected and measured using, for example, a competitive RIA cGMP kit (Perkin-Elmer, Boston, Mass.).

The activity of a NP also can be evaluated in vivo by, for example, testing its effects on factors such as pulmonary capillary wedge pressure, right atrial pressure, mean arterial pressure, urinary sodium excretion, urine flow, proximal and distal fractional sodium reabsorption, plasma renin activity, plasma cGMP levels, urinary cGMP excretion, net renal generation of cGMP, glomerular filtration rate, and left ventricular mass in animals. In some cases, such parameters can be evaluated after induced MI (e.g., MI induced by coronary artery ligation).

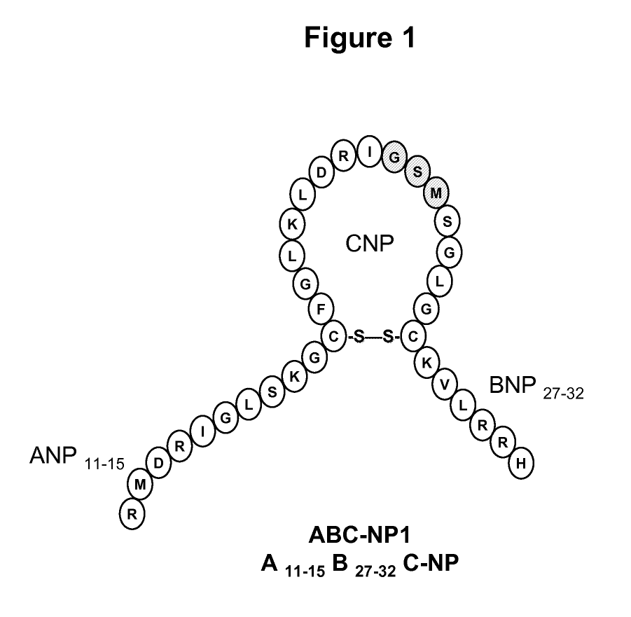

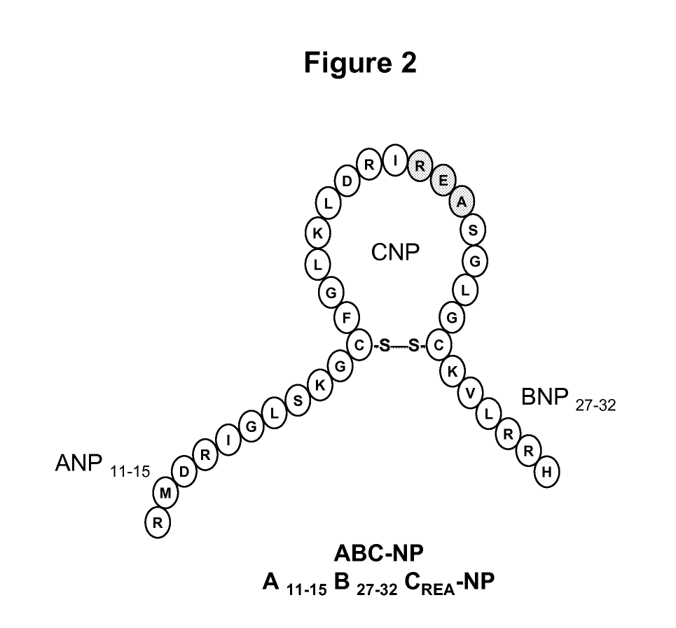

The NPs provided herein typically are cyclic due to disulfide bonds between cysteine residues (see, e.g., the structures depicted in FIGS. 1 and 2). In some embodiments, a sulfhydryl group on a cysteine residue can be replaced with an alternative group (e.g., —CH₂CH₂—). To replace a sulfhydryl group with a —CH₂— group, for example, a cysteine residue can be replaced by alpha-aminobutyric acid. Such cyclic analog polypeptides can be generated, for example, in accordance with the methodology of Lebl and Hruby ((1984) Tetrahedron Lett. 25:2067), or by employing the procedure disclosed in U.S. Pat. No. 4,161,521.

In addition, ester or amide bridges can be formed by reacting the OH of serine or threonine with the carboxyl group of aspartic acid or glutamic acid to yield a bridge having the structure —CH₂CO₂CH₂—. Similarly, an amide can be obtained by reacting the side chain of lysine with aspartic acid or glutamic acid to yield a bridge having the structure —CH₂C(O)NH(CH)₄—. Methods for synthesis of these bridges are known in the art (see, e.g., Schiller et al. (1985) Biochem. Biophy. Res. Comm. 127:558, and Schiller et al. (1985) Int. J. Peptide Protein Res. 25:171). Other bridge-forming amino acid residues and reactions are provided in, for example, U.S. Pat. No. 4,935,492. Preparation of peptide analogs that include non-peptidyl bonds to link amino acid residues also are known in the art. See, e.g., Spatola et al. (1986) Life Sci. 38:1243; Spatola (1983) Vega Data 1(3); Morley (1980) Trends Pharm. Sci. 463-468; Hudson et al. (1979) Int. J. Pept. Prot. Res. 14:177; Spatola, in Chemistry and Biochemistry of Amino Acid Peptides and Proteins, B. Weinstein, ed., Marcel Dekker, New York, p. 267 (1983); Hann (1982) J. Chem. Soc. Perkin Trans. 1:307; Almquist et al. (1980) J. Med. Chem. 23:1392; Jennings-White et al. (1982) Tetrahedron Lett. 23:2533; European Patent Application EP 45665; Holladay et al. (1983) Tetrahedron Lett. 24:4401; and Hruby (1982) Life Sci. 31:189.

In some embodiments, a NP can comprise an amino acid sequence as set forth in SEQ ID NOS:5, 23, 25, 30, 34, 36, 38, 40, 43, 46, 48, or 51, but with a particular number of amino acid substitutions. For example, a NP can have the amino acid sequence of any one of SEQ ID NOS:5, 23, 25, 30, 34, 36, 38, 40, 43, 46, 48, or 51, but with one, two, three, four, or five amino acid substitutions. Examples of such amino acid sequences include, without limitation, those set forth in SEQ ID NOS:52-163.

In some embodiments, a NP as provided herein can include an amino acid sequence with at least 85% (e.g., 85%, 86%, 87%, 88%, 89%, 90%, 91%, 92%, 93%, 94%, 95%, 96%, 97%, 97.5%, 98%, 98.5%, 99.0%, 99.5%, 99.6%, 99.7%, 99.8%, 99.9%, or 100%) sequence identity to a region of a reference NP sequence (e.g., any one of SEQ ID NOS:1-51). Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

Percent sequence identity is determined by comparing a target nucleic acid or amino acid sequence to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained on the World Wide Web from Fish & Richardson's web site (fr.com/blast) or the U.S. government's National Center for Biotechnology Information web site (ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\Bl2seq c:\seq1.txt-j c:\seq2.txt-p blastn-o c:\output.txt-q-1-r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a target sequence that is 30 amino acids in length is compared to the sequence set forth in SEQ ID NO:1, (2) the Bl2seq program presents 27 amino acids from the target sequence aligned with a region of the sequence set forth in SEQ ID NO:1 where the first and last amino acids of that 27 amino acid region are matches, and (3) the number of matches over those 27 aligned amino acids is 25, then the 30 amino acid target sequence contains a length of 27 and a percent identity over that length of 92.6 (i.e., 25÷27×100=92.6).

It will be appreciated that different regions within a single amino acid or nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Isolated polypeptides can be produced using any suitable methods, including solid phase synthesis, and can be generated using manual techniques or automated techniques (e.g., using an Applied BioSystems (Foster City, Calif.) Peptide Synthesizer or a Biosearch Inc. (San Rafael, Calif.) automatic peptide synthesizer. Disulfide bonds between cysteine residues can be introduced by mild oxidation of the linear polypeptides using KCN as taught, e.g., in U.S. Pat. No. 4,757,048. NPs also can be produced recombinantly, as described herein.

In some cases, a polypeptide provided herein can be a substantially pure polypeptide. As used herein, the term “substantially pure” with reference to a polypeptide means that the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure or is any chemically synthesized polypeptide. A substantially pure polypeptide can be at least about 60, 65, 70, 75, 80, 85, 90, 95, or 99 percent pure. Typically, a substantially pure polypeptide will yield a single major band on a non-reducing polyacrylamide gel.

Salts of carboxyl groups of polypeptides can be prepared by contacting the peptide with one or more equivalents of a desired base such as, for example, a metallic hydroxide base (e.g., sodium hydroxide), a metal carbonate or bicarbonate base (e.g., sodium carbonate or sodium bicarbonate), or an amine base (e.g., triethylamine, triethanolamine, and the like). Acid addition salts of polypeptides can be prepared by contacting the polypeptide with one or more equivalents of an inorganic or organic acid (e.g., hydrochloric acid).

Esters of carboxyl groups of polypeptides can be prepared using any suitable means (e.g., those known in the art) for converting a carboxylic acid or precursor to an ester. For example, one method for preparing esters of the present polypeptides, when using the Merrifield synthesis technique, is to cleave the completed polypeptide from the resin in the presence of the desired alcohol under either basic or acidic conditions, depending upon the resin. The C-terminal end of the polypeptide then can be directly esterified when freed from the resin, without isolation of the free acid.

Amides of polypeptides can be prepared using techniques (e.g., those known in the art) for converting a carboxylic acid group or precursor to an amide. One method for amide formation at the C-terminal carboxyl group includes cleaving the polypeptide from a solid support with an appropriate amine, or cleaving in the presence of an alcohol, yielding an ester, followed by aminolysis with the desired amine.

N-acyl derivatives of an amino group of a polypeptide can be prepared by utilizing an N-acyl protected amino acid for the final condensation, or by acylating a protected or unprotected peptide. O-acyl derivatives can be prepared for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

In some embodiments, the NPs provided herein can have half-lives that are increased relative to the half-life of native NPs. For example, while the half-life of CNP after administration to a mammal is short (about a minute and a half), the elimination half-life of a chimeric NP can be increased. A NP provided herein can have a half life that is increased by at least 2-fold (e.g., at least 2-fold, at least 3-fold, at least 4-fold, at least 5-fold, at least 6-fold, at least 7-fold, at least 8-fold, at least 9-fold, or at least 10-fold) as compared to a native NP such as CNP, for example. In some embodiments, a NP can have an elimination half-life of at least about 10 minutes (e.g., at least about 10 minutes, at least about 12 minutes, at least about 15 minutes, at least about 17 minutes, at least about 18 minutes, or at least about 20 minutes).

In some embodiments, a NP can be modified by linkage to a polymer such as polyethylene glycol (PEG), or by fusion to another polypeptide such as albumin, for example. In some embodiments, one or more PEG moieties can be conjugated to a NP via lysine residues. Linkage to PEG or another suitable polymer, or fusion to albumin or another suitable polypeptide can result in a modified NP having an increased half life as compared to an unmodified NP. Without being bound by a particular mechanism, an increased serum half life can result from reduced proteolytic degradation, immune recognition, or cell scavanging of the modified NP. Methods for modifying a polypeptide by linkage to PEG (also referred to as “PEGylation”) or other polymers are known in the art, and include those set forth in U.S. Pat. No. 6,884,780; Cataliotti et al. ((2007) Trends Cardiovasc. Med. 17:10-14; Veronese and Mero (2008) BioDrugs 22:315-329; Miller et al. (2006) Bioconjugate Chem. 17:267-274; and Veronese and Pasut (2005) Drug Discov. Today 10:1451-1458, all of which are incorporated herein by reference in their entirety. Methods for modifying a polypeptide by fusion to albumin also are known in the art, and include those set forth in U.S. Patent Publication No. 20040086976, and Wang et al. (2004) Pharm. Res. 21:2105-2111, both of which are incorporated herein by reference in their entirety.

A NP as provided herein can function through one or more of the guanylyl cyclase receptors through which the native NPs function. For example, in some embodiments, a NP as provided herein can bind to and function through the NPR-A receptor through which ANP and BNP function. In some cases, a NP as provided herein can function through the NPR-B receptor through which CNP functions. Further, in some cases, a NP as provided herein can bind to and function through more than one guanylyl cyclase receptor, including NPR-A and NPR-B, for example. Methods for evaluating which receptor is involved in function of a particular NP are known in the art. For example, glomeruli, which contain both NPR-A and NPR-B, can be isolated (e.g., from a laboratory animal such as a dog) and incubated with a NP (e.g., a native, chimeric, or mutated NP), and cGMP levels can be measured. Glomeruli can be pretreated with antagonists of NPR-A or NPR-B to determine whether cGMP production stimulated by a NP through one or the other receptor can be attenuated.

In some cases, a compound (e.g., an isolated NP) provided herein can reduce or prevent restenosis. The presence or extent of restenosis can be evaluated using methods known in the art, including, for example, angiogram. In some cases, a compound (e.g., an isolated NP) provided herein can reduce or prevent cardiac remodeling. The term “cardiac remodeling” refers to effects on the heart that can occur with MI, acute heart failure (AHF), or other conditions. These include, for example, heart dilation, myocyte hypertrophy, and cardiofibrosis (i.e., proliferation of interstitial fibroblasts). The NPs provided herein can inhibit or prevent cardiac remodeling that occurs with acute MI or AHF. In some embodiments, parameters indicative of reduced cardiac remodeling can include one or more of the following: cardiac unloading (i.e., reduced pressure in the heart), increased glomerular filtration rate (GFR), decreased plasma renin activity (PRA), decreased levels of angiotensin II, decreased proliferation of cardiac fibroblasts, decreased left ventricular (LV) hypertrophy), decreased LV mass (indicative of reduced fibrosis and hypertrophy), decreased pulmonary wedge capillary pressure (PWCP; an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter. To determine whether a NP is capable of inhibiting or reducing cardiac remodeling, one or more of these parameters can be evaluated (e.g., before and after treatment with the NP), using methods known in the art, for example.

### Nucleic Acids, Vectors, and Host Cells

This document also describes exemplary nucleic acids encoding polypeptides (e.g., NPs), as well as expression vectors containing the nucleic acids, and host cells containing the nucleic acids and/or expression vectors. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding the NPs, variant NPs, and chimeric NPs provided herein.

An “isolated nucleic acid” is a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid. By way of example and not limitation, an “isolated ANP nucleic acid,” for example, can be a RNA or DNA molecule containing 9 or more (e.g., 15 or more, 21 or more, 36 or more, or 45 or more) sequential nucleotide bases that encode at least a portion of ANP, or a RNA or DNA complementary thereto.

Also provided herein are nucleic acid molecules that can selectively hybridize under stringent hybridization conditions to a nucleic acid molecule encoding a NP (e.g., a nucleic acid molecule encoding a polypeptide having the amino acid sequence set forth in any of SEQ ID NOS:5, 23, 25, 30, 34, 36, 38, 40, 43, 46, 48, or 51), or variants and fragments thereof. The term “selectively hybridize” means to detectably and specifically bind under hybridization and wash conditions that minimize appreciable amounts of detectable binding to nonspecific nucleic acids. For example, high stringency conditions can be used to achieve selective hybridization conditions. Moderate and stringent hybridization conditions include those that are well known in the art. See, for example, sections 9.47-9.51 of Sambrook et al. (1989). As used herein, stringent conditions are those that (1) employ low ionic strength and high temperature for washing, such as 0.015 M NaCl/0.0015 M sodium citrate (SSC) with 0.1% sodium lauryl sulfate (SDS) at 50° C., or (2) employ a denaturing agent such as formamide during hybridization, such as 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Alternatively, 50% formamide, 5×SSC (0.75 M NaCl, 0.075 M sodium citrate), 50 mM sodium phosphate (pH 6.8), 0.1% sodium phosphate, 5×Denhardt's solution, sonicated salmon sperm DNA (50 μg/ml), 0.1% sodium dodecylsulfate (SDS), and 10% dextran sulfate at 42° C. can be used, with washes at 42° C. in 0.2×SSC and 0.1% SDS.

Isolated nucleic acid molecules can be produced using standard techniques, including, without limitation, common molecular cloning and chemical nucleic acid synthesis techniques. For example, polymerase chain reaction (PCR) techniques can be used to obtain an isolated nucleic acid containing nucleotide sequence that encodes a NP as provided herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids (e.g., nucleic acids encoding variant NPs) also can be obtained by mutagenesis. For example, a reference sequence can be mutated using standard techniques including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992. Non-limiting examples of variant NPs art provided herein.

This document also contemplates nucleic acid molecules encoding amino acid sequences from NPs other than ANP, BNP, CNP, DNP, URO, or chimeras or variants thereof. Sources of nucleotide sequences from which nucleic acid molecules encoding a NP, or the nucleic acid complement thereof, can be obtained include total or polyA+ RNA from any eukaryotic source, including reptilian (e.g., snake) or mammalian (e.g., human, rat, mouse, canine, bovine, equine, ovine, caprine, or feline) cellular source from which cDNAs can be derived by methods known in the art. Other sources of the nucleic acid molecules provided herein include genomic libraries derived from any eukaryotic cellular source, including mammalian sources as exemplified above.

Nucleic acid molecules encoding native NPs can be identified and isolated using standard methods, e.g., as described by Sambrook et al., Molecular Cloning: A Laboratory Manual, Cold Spring Harbor Laboratory Press, NY (1989). For example, reverse-transcriptase PCR (RT-PCR) can be used to isolate and clone NP cDNAs from isolated RNA that contains RNA sequences of interest (e.g., total RNA isolated from human tissue). Other approaches to identify, isolate and clone NP cDNAs include, for example, screening cDNA libraries.

Vectors containing nucleic acids such as those described herein also are provided. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In the expression vectors provided herein, a nucleic acid (e.g., a nucleic acid encoding a NP) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors thus can be useful to produce antibodies as well as other multivalent molecules.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

Host cells containing vectors also are provided. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Suitable methods for transforming and transfecting host cells can be found, for example, in Sambrook et al., Molecular Cloning: A Laboratory Manual (2^{nd} edition), Cold Spring Harbor Laboratory, New York (1989). For example, calcium phosphate precipitation, electroporation, heat shock, lipofection, microinjection, and viral-mediated nucleic acid transfer can be used introduce nucleic acid into cells. In addition, naked DNA can be delivered directly to cells in vivo as described elsewhere (U.S. Pat. Nos. 5,580,859 and 5,589,466).

### Detecting Polypeptides

This document provides methods and materials for detecting a polypeptide provided herein. Such methods and materials can be used to monitor polypeptide levels within a mammal receiving the polypeptide as a therapeutic. A NP provided herein can be detected, for example, immunologically, using one or more antibodies. As used herein, the term “antibody” includes intact molecules as well as fragments thereof that are capable of binding to an epitopic determinant of a polypeptide provided herein. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The term “antibody” includes polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are contained in the sera of the immunized animals. Monoclonal antibodies are homogeneous populations of antibodies to a particular epitope of an antigen.

Antibody fragments that have specific binding affinity for a NP provided herein can be generated by known techniques. For example, F(ab′)2 fragments can be produced by pepsin digestion of the antibody molecule; Fab fragments can be generated by reducing the disulfide bridges of F(ab′)2 fragments. In some cases, Fab expression libraries can be constructed. See, for example, Huse et al., Science, 246:1275 (1989). Once produced, antibodies or fragments thereof can be tested for recognition of a polypeptide provided herein by standard immunoassay methods including ELISA techniques, radioimmunoassays, and Western blotting. See, Short Protocols in Molecular Biology, Chapter 11, Green Publishing Associates and John Wiley & Sons, Ed. Ausubel et al., 1992.

In immunological assays, an antibody having specific binding affinity for a polypeptide provided herein or a secondary antibody that binds to such an antibody can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, PerCP, rhodamine, or PE), luminescent moieties (e.g., Qdot™ nanoparticles supplied by Invitrogen (Carlsbad, Calif.)), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting a polypeptide provided herein can be performed in a variety of known formats, including sandwich assays, competition assays (competitive RIA), or bridge immunoassays. See, for example, U.S. Pat. Nos. 5,296,347; 4,233,402; 4,098,876; and 4,034,074. Methods of detecting a polypeptide provided herein generally include contacting a biological sample with an antibody that binds to a polypeptide provided herein and detecting binding of the polypeptide to the antibody. For example, an antibody having specific binding affinity for a polypeptide provided herein can be immobilized on a solid substrate by any of a variety of methods known in the art and then exposed to the biological sample. Binding of the polypeptide to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden). In some cases, the antibody can be labeled and detected as described above. A standard curve using known quantities of a polypeptide provided herein can be generated to aid in the quantitation of the levels of the polypeptide.

In some embodiments, a “sandwich” assay in which a capture antibody is immobilized on a solid substrate can be used to detect the presence, absence, or level of a polypeptide provided herein. The solid substrate can be contacted with the biological sample such that any polypeptide of interest in the sample can bind to the immobilized antibody. The presence, absence, or level of the polypeptide bound to the antibody can be determined using a “detection” antibody having specific binding affinity for the polypeptide. In some embodiments, a capture antibody can be used that has binding affinity for ANP, BNP, CNP, DNP, and/or URO, or a chimeric or variant polypeptide as described herein, and a detection antibody can be used that has specific binding affinity for a particular polypeptide provided herein (e.g., a polypeptide having the amino acid sequence set forth in any of SEQ ID NOS:5, 23, 25, 30, 34, 36, 38, 40, 43, 46, 48, or 51). It is understood that in sandwich assays, the capture antibody should not bind to the same epitope (or range of epitopes in the case of a polyclonal antibody) as the detection antibody. Thus, if a monoclonal antibody is used as a capture antibody, the detection antibody can be another monoclonal antibody that binds to an epitope that is either physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the detection antibody can be either a monoclonal antibody that binds to an epitope that is either physically separated from or partially overlaps with any of the epitopes to which the capture polyclonal antibody binds, or a polyclonal antibody that binds to epitopes other than or in addition to that to which the capture polyclonal antibody binds. Sandwich assays can be performed as sandwich ELISA assays, sandwich Western blotting assays, or sandwich immunomagnetic detection assays.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used.

Antibodies having specific binding affinity for a polypeptide provided herein can be produced through standard methods. For example, a polypeptide can be recombinantly produced as described above, can be purified from a biological sample (e.g., a heterologous expression system), or can be chemically synthesized, and used to immunize host animals, including rabbits, chickens, mice, guinea pigs, or rats. For example, a polypeptide having the amino acid sequence set forth in any of SEQ ID NOS:5, 23, 25, 30, 34, 36, 38, 40, 43, 46, 48, or 51, or fragments or variants thereof that are at least six amino acids in length, can be used to immunize an animal. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Monoclonal antibodies can be prepared using a polypeptide provided herein and standard hybridoma technology. In particular, monoclonal antibodies can be obtained by any technique that provides for the production of antibody molecules by continuous cell lines in culture such as described by Kohler et al., Nature, 256:495 (1975), the human B-cell hybridoma technique (Kosbor et al., Immunology Today, 4:72 (1983); Cote et al., Proc. Natl. Acad. Sci. USA, 80:2026 (1983)), and the EBV-hybridoma technique (Cole et al., “Monoclonal Antibodies and Cancer Therapy,” Alan R. Liss, Inc., pp. 77-96 (1983)). Such antibodies can be of any immunoglobulin class including IgG, IgM, IgE, IgA, IgD, and any subclass thereof. The hybridoma producing the monoclonal antibodies can be cultivated in vitro and in vivo.

Other techniques for detecting a polypeptide provided herein include mass-spectrophotometric techniques such as electrospray ionization (ESI), and matrix-assisted laser desorption-ionization (MALDI). See, for example, Gevaert et al., Electrophoresis, 22:1645-51 (2001); Chaurand et al., J. Am. Soc. Mass Spectrom., 10:91-103 (1999). Mass spectrometers useful for such applications are available from Applied Biosystems (Foster City, Calif.); Bruker Daltronics (Billerica, Mass.); and Amersham Pharmacia (Sunnyvale, Calif.).

### Compositions and Methods for Administration

The compounds described herein (e.g., native NPs, as well as chimeric and variant NPs), or nucleic acids encoding the polypeptides described herein, can be incorporated into compositions for administration to a subject (e.g., a subject suffering from or at risk for restenosis). Methods for formulating and subsequently administering therapeutic compositions are well known to those in the art. Dosages typically are dependent on the responsiveness of the subject to the compound, with the course of treatment lasting from several days to several months, or until a suitable response is achieved. Persons of ordinary skill in the art routinely determine optimum dosages, dosing methodologies and repetition rates. Optimum dosages can vary depending on the relative potency of an antibody, and generally can be estimated based on the EC₅₀ found to be effective in in vitro and/or in vivo animal models. Compositions containing the compounds (e.g., NPs) and nucleic acids provided herein may be given once or more daily, weekly, monthly, or even less often, or can be administered continuously for a period of time (e.g., hours, days, or weeks). For example, a NP or a composition containing a NP can be administered to a MI patient at a dose of at least about 0.01 ng NP/kg to about 100 mg NP/kg of body mass at or about the time of reperfusion, or can be administered continuously as an infusion beginning at or about the time of reperfusion and continuing for one to seven days (e.g., at a dose of about 0.01 ng NP/kg/minute to about 0.5 μg NP/kg/minute).

This document also provides for the use of the NPs and nucleic acids disclosed herein in the manufacture of medicaments (e.g., for increasing natriuretic activity within a mammal, for treating a mammal having a cardiovascular condition or a renal condition, for reducing cardiac remodeling in a subject identified as being in need thereof, or for reducing restenosis. The NPs and nucleic acids can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption.

In some embodiments, a composition can contain a NP as provided herein in combination with a pharmaceutically acceptable carrier. Pharmaceutically acceptable carriers include, for example, pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions containing molecules described herein can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

Compositions and formulations for parenteral, intrathecal or intraventricular administration include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Formulations for topical administration include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful. In some embodiments, transdermal delivery of NPs as provided herein can be particularly useful. Methods and compositions for transdermal delivery include those described in the art (e.g., in Wermeling et al. (2008) Proc. Natl. Acad. Sci. USA 105:2058-2063; Goebel and Neubert (2008) Skin Pharmacol. Physiol. 21:3-9; Banga (2007) Pharm. Res. 24:1357-1359; Malik et al. (2007) Curr. Drug Deliv. 4:141-151; and Prausnitz (2006) Nat. Biotechnol. 24:416-417).

Nasal preparations can be presented in a liquid form or as a dry product. Nebulized aqueous suspensions or solutions can include carriers or excipients to adjust pH and/or tonicity.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsion formulations are particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

Compositions provided herein can contain any pharmaceutically acceptable salts, esters, or salts of such esters, or any other compound which, upon administration to a subject, is capable of providing (directly or indirectly) the biologically active metabolite or residue thereof for the relevant compound (e.g., NP). Accordingly, for example, this document describes pharmaceutically acceptable salts of NPs, prodrugs and pharmaceutically acceptable salts of such prodrugs, and other bioequivalents. A prodrug is a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the NPs useful in methods provided herein (i.e., salts that retain the desired biological activity of the parent NPs without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed with elemental anions (e.g., bromine, iodine, or chlorine).

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the other components within the compositions.

In some cases, a polypeptide provided herein can be formulated as a sustained release dosage form. For example, a NP can be formulated into a controlled release formulation. In some cases, coatings, envelopes, or protective matrices can be formulated to contain one or more of the polypeptides provided herein. Such coatings, envelopes, and protective matrices can be used to coat indwelling devices such as stents, catheters, and peritoneal dialysis tubing. In some cases, a polypeptide provided herein can incorporated into a polymeric substances, liposomes, microemulsions, microparticles, nanoparticles, or waxes.

Pharmaceutical formulations as disclosed herein, which can be presented conveniently in unit dosage form, can be prepared according to conventional techniques well known in the pharmaceutical industry. Such techniques include the step of bringing into association the active ingredients (i.e., the antibodies) with the desired pharmaceutical carrier(s). Typically, the formulations can be prepared by uniformly and intimately bringing the active ingredients into association with liquid carriers or finely divided solid carriers or both, and then, if necessary, shaping the product. Formulations can be sterilized if desired, provided that the method of sterilization does not interfere with the effectiveness of the molecules(s) contained in the formulation.

### Methods

This document also provides methods for using compounds (e.g., NPs) as disclosed herein for increasing natriuresis and diuresis, for reducing or preventing restenosis, and/or for reducing or preventing cardiac remodeling. In some embodiments, for example, the compounds and nucleic acid molecules described herein can be administered to a mammal (e.g., a human or a non-human mammal) to treat cardiovascular conditions (e.g., acute decompensated heart failure, acute coronary syndromes, and ventricular remodeling post-myocardial infarction) and/or renal conditions (e.g., perioperative renal dysfunction, renal dysfunction secondary to heart failure, and diabetic nephropathy). In some cases, the compounds and nucleic acid molecules provided herein can be administered to a mammal (e.g., a human or a non-human mammal) in order to reduce or prevent restenosis that can occur, for example, after angioplasty, stent placement, vascular surgery, cardiac surgery, or interventional radiology. In some cases, the compounds and nucleic acid molecules described herein can be administered to a mammal (e.g., a human or a non-human mammal) in order to reduce or inhibit cardiac remodeling that can occur, for example, after MI.

The composition or NP can be administered at any suitable dose, depending on various factors including, without limitation, the agent chosen and the patient characteristics. Administration can be local or systemic.

In some embodiments, a NP or a composition containing a NP can be administered at a dose of at least about 0.01 ng NP/kg to about 100 mg NP/kg of body mass (e.g., about 10 ng NP/kg to about 50 mg NP/kg, about 20 ng NP/kg to about 10 mg NP/kg, about 0.1 ng NP/kg to about 20 ng NP/kg, about 3 ng NP/kg to about 10 ng NP/kg, or about 50 ng NP/kg to about 100 μg/kg) of body mass, although other dosages also may provide beneficial results. A composition can be administered at a dose of, for example, about 0.1 ng NP/kg/minute to about 500 ng NP/kg/minute (e.g., about 0.5 ng NP/kg/minute, about 1 ng NP/kg/minute, about 2 ng NP/kg/minute, about 3 ng NP/kg/minute, about 5 ng NP/kg/minute, about 7.5 ng NP/kg/minute, about 10 ng NP/kg/minute, about 12.5 ng NP/kg/minute, about 15 ng NP/kg/minute, about 20 ng NP/kg/minute, about 25 ng NP/kg/minute, about 30 ng NP/kg/minute, about 50 ng NP/kg/minute, about 100 ng NP/kg/minute, or about 300 ng NP/kg/minute). In some cases, when a NP is to be administered after MI, for example, a composition containing the NP can be administered as a continuous intravenous infusion beginning at or about the time of reperfusion (i.e., at the time an occluded artery is opened), and continuing for one to seven days (e.g., one, two, three, four, five, six, or seven days). In some embodiments, a composition containing a NP can be administered before reperfusion (e.g., about one hour prior to reperfusion), either as one or more individual doses or as a continuous infusion beginning about one hour prior to reperfusion). For example, a composition can be administered beginning about one hour, about 45 minutes, about 30 minutes, or about 15 minutes prior to reperfusion. In some cases, a composition containing a NP as provided herein can be administered after reperfusion (e.g., within about ten hours of reperfusion), and can be administered either as one or more individual doses or as a continuous infusion beginning within about ten hours of reperfusion. For example, a composition can be administered about one hour, about two hours, about three hours, about four hours, about five hours, about six hours, about seven hours, about eight hours, about nine hours, or about ten hours after reperfusion.

In some embodiments, a NP or a composition containing a NP can be administered via a first route (e.g., intravenously) for a first period of time, and then can be administered via another route (e.g., topically or subcutaneously) for a second period of time. For example, a composition containing a NP can be intravenously administered to a mammal (e.g., a human) at a dose of about 0.1 ng NP/kg/minute to about 300 ng NP/kg/minute (e.g., about 1 ng NP/kg/minute to about 15 ng NP/kg/minute, about 3 ng NP/kg/minute to about 10 ng NP/kg/minute, or about 10 ng NP/kg/minute to about 30 ng NP/kg/minute) for one to seven days (e.g., one, two, three, four, five, six, or seven days), and subsequently can be subcutaneously administered to the mammal at a dose of about 10 ng NP/kg/day to about 100 ng NP/kg/day (e.g., about 10 ng NP/kg/day, about 20 ng NP/kg/day, about 25 ng NP/kg/day, about 30 ng NP/kg/day, about 50 ng NP/kg/day, or about 100 ng NP/kg/day) for five to 30 days (e.g., seven, 10, 14, 18, 21, 24, or 27 days).

The methods provided herein can include administering to a mammal an effective amount of a NP (e.g., a native, chimeric, or variant NP) or a nucleic acid encoding a NP, or an effective amount of a composition containing such a molecule. As used herein, the term “effective amount” is an amount of a molecule or composition that is sufficient to alter the desired parameter by at least 10%. For example, in some embodiments, an “effective amount” of a NP can be an amount of the NP that is sufficient to increase natriuresis and/or diuresis (or a characteristic of natriuresis and/or diuresis such as plasma cGMP levels, urinary cGMP excretion, net renal cGMP generation, urine flow, urinary sodium excretion, urinary potassium excretion, hematocrit, plasma BNP immunoreactivity, renal blood flow, plasma ANP immunoreactivity, renal vascular resistance, proximal and distal fractional reabsorption of sodium, mean arterial pressure, pulmonary capillary wedge pressure, right atrial pressure, pulmonary arterial pressure, plasma renin activity, plasma angiotensin II levels, plasma aldosterone levels, renal perfusion pressure, and systemic vascular resistance) by at least 10% (e.g., 10%, 15%, 20%, 25%, 30%, 40%, 50%, 60%, 70%, 75%, 80%, 85%, 90%, 95%, 99%, or 100%). For example, an “effective amount” of a NP can be an amount that increases sodium excretion in a treated mammal by at least 10% as compared to the level of sodium excretion in the mammal prior to administration of the NP, or as compared to the level of sodium excretion in a control, untreated mammal.

In some embodiments, an “effective amount” of a NP can be an amount of the NP that is sufficient to reduce the occurrence of restenosis in a mammalian recipient by at least 10% (e.g., 10%, 15%, 20%, 25%, 30%, 40%, 50%, 60%, 70%, 75%, 80%, 85%, 90%, 95%, 99%, or 100%). In some cases, for example, an “effective amount” of a NP as provided herein can be an amount that reduces restenosis in a treated mammal by at least 10% as compared to the level of restenosis in the mammal prior to administration of the NP or without administration of the NP (e.g., the level of restenosis after a previous angioplasty or stent placement procedure for example), or as compared to the level of restenosis in a control, untreated mammal. The presence or extent of restenosis can be evaluated using methods known in the art, including, for example, angiogram.

In some embodiments, an “effective amount” of a NP can be an amount of the NP that is sufficient to reduce the occurrence of cardiac remodeling in a mammalian recipient by at least 10% (e.g., 10%, 15%, 20%, 25%, 30%, 40%, 50%, 60%, 70%, 75%, 80%, 85%, 90%, 95%, 99%, or 100%). For example, an effective amount of a NP as provided herein can increase ejection fraction, GFR, urinary sodium excretion, or urine flow by at least 10%, and/or can decrease PRA, LV mass, cardiac fibroblast proliferation, PWCP, RAP, MAP, aldosterone levels, LV hypertrophy, ventricular fibrosis, LV end systolic diameter, proximal fractional sodium reabsorption, or distal fractional sodium reabsorption by at least 10%, and/or can result in cardiac unloading.

The invention will be further described in the following example, which does not limit the scope of the invention described in the claims.

### EXAMPLES

### Example 1

A five amino acid sequence from ANP was fused to the N-terminus of CNP, and a six amino acid sequence from BNP was fused to the C-terminus of CNP. This novel hybrid peptide, termed ABC-NP1 (FIG. 1), was previously shown to have cardiac unloading actions and mild hypotensive effects, similar to CNP. Importantly however, the N- and C-terminal alterations resulted in potent renal excretory actions. To test the hypothesis that amino acids GSM15-17 in the CNP disulfide-ring mediate the vascular and hypotensive actions of ABC-NP1, GSM₁₅₋₁₇ was mutated to REA₁₅₋₁₇. The mutated polypeptide was named ABC-NP (FIG. 2), and its in vivo and in vitro actions were compared to those of ABC-NP.

Cardiorenal and humoral actions of intravenous bolus administration of ABC-NP1 (n=5) and ABC-NP (n=5) at 25 microgram/Kg were evaluated in two separate groups of normal anesthetized dogs. The cGMP response of both peptides also was assessed in HAEC, HCF, and isolated canine glomeruli.

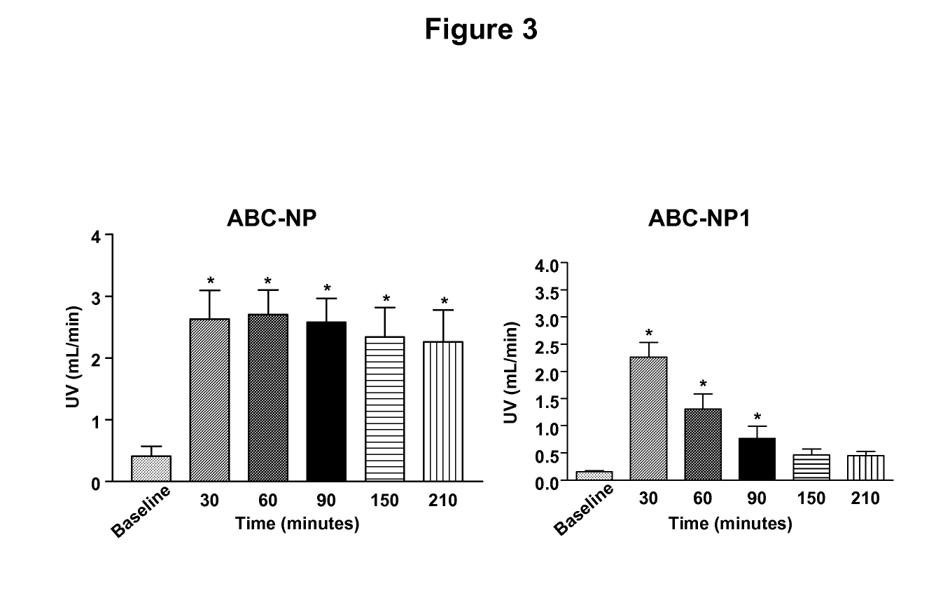

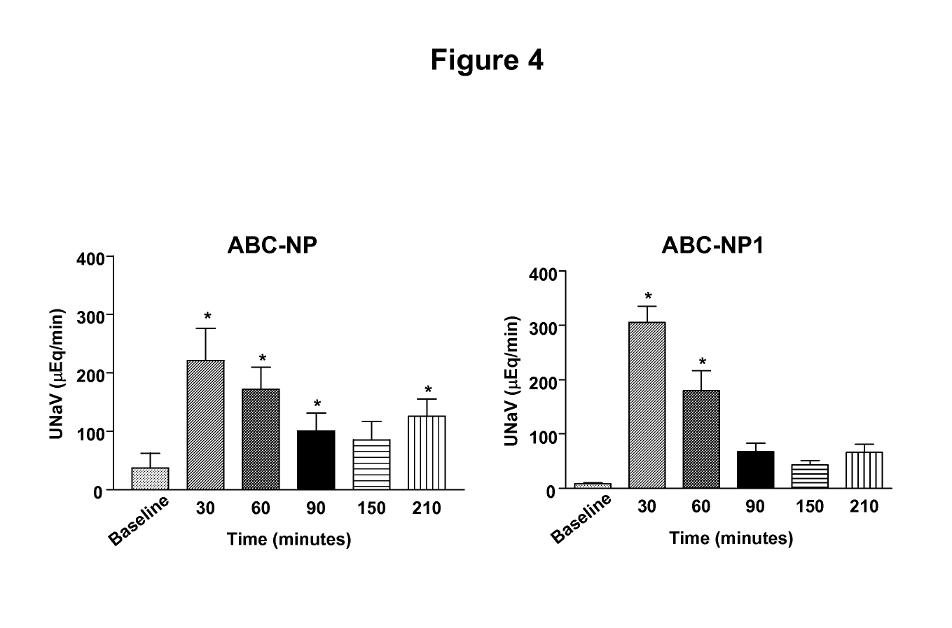

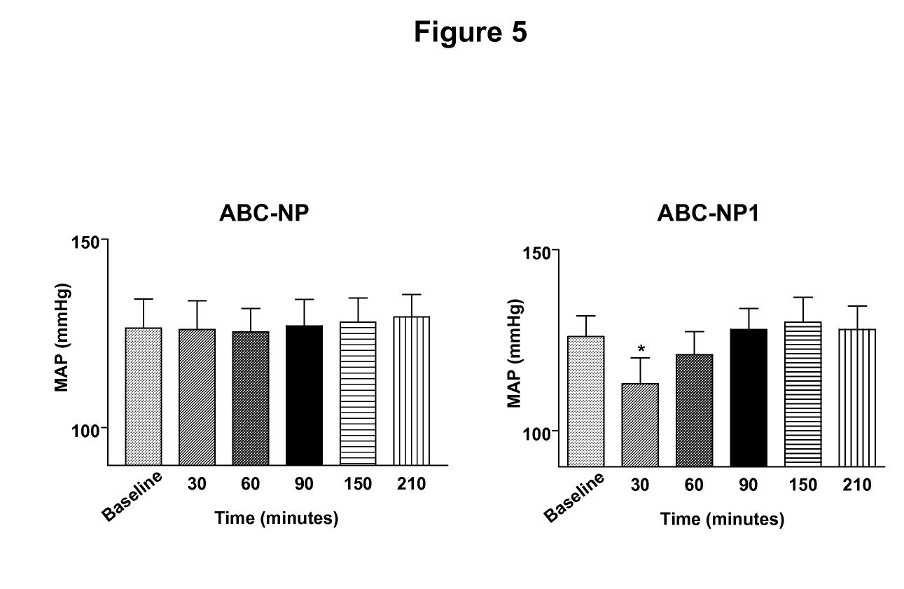

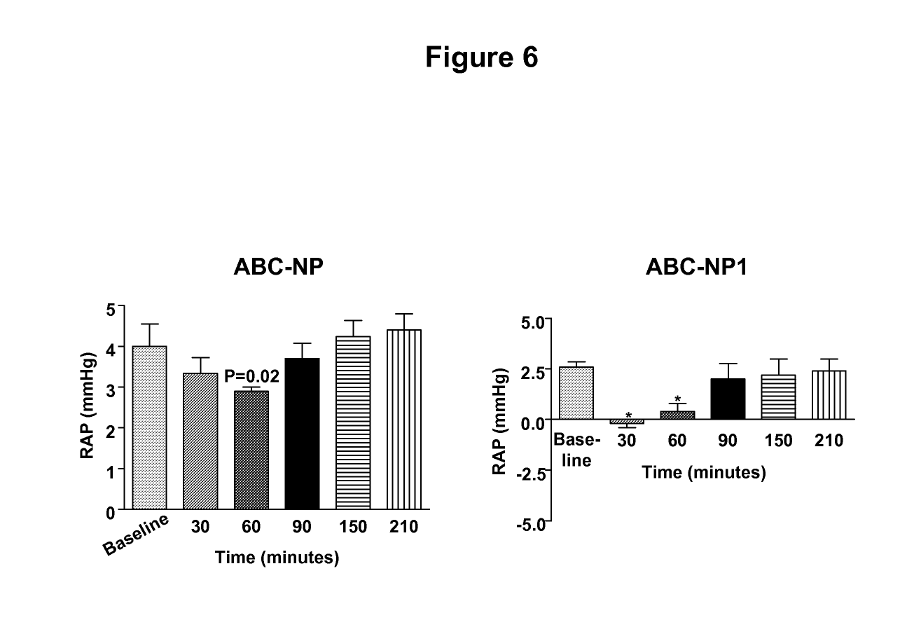

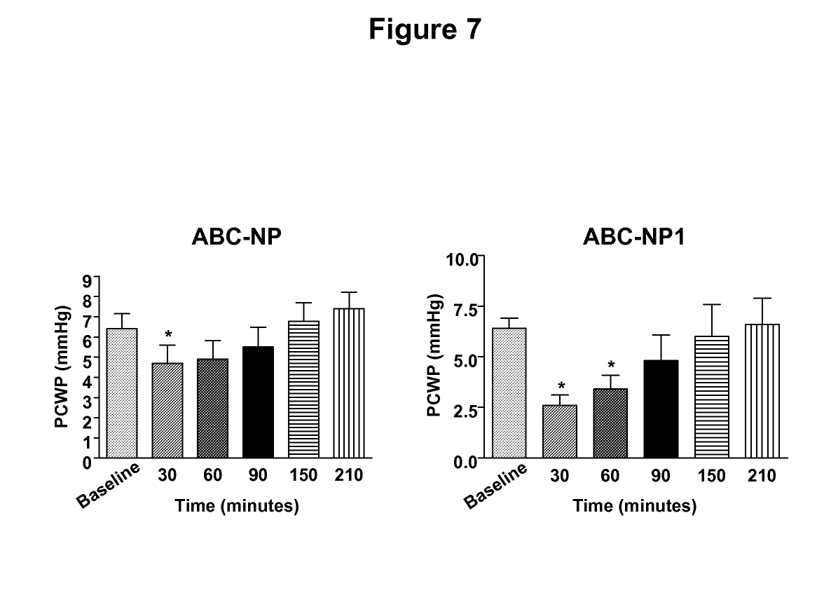

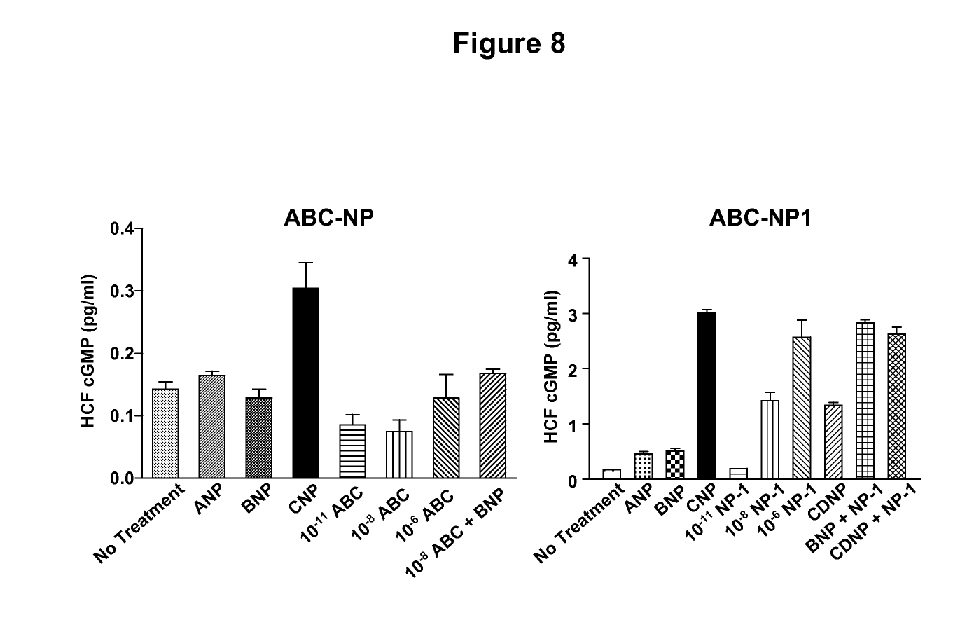

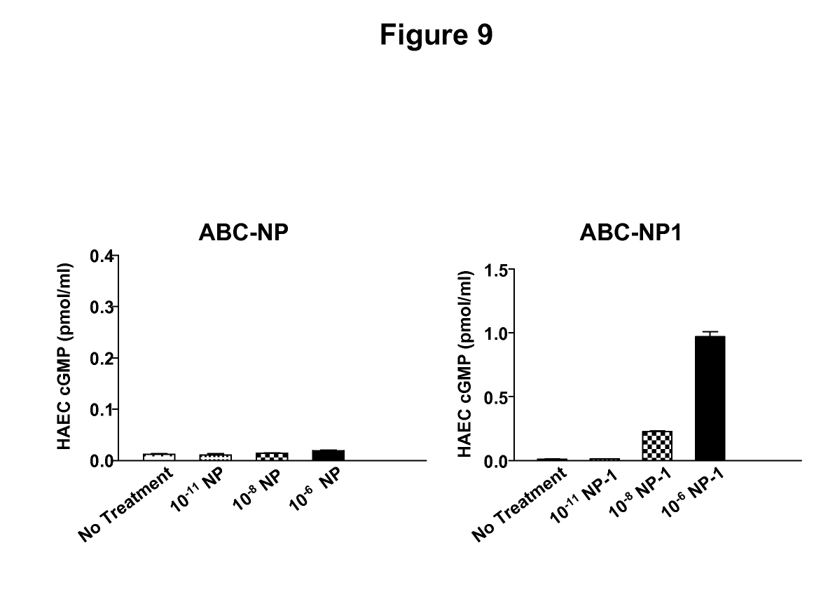

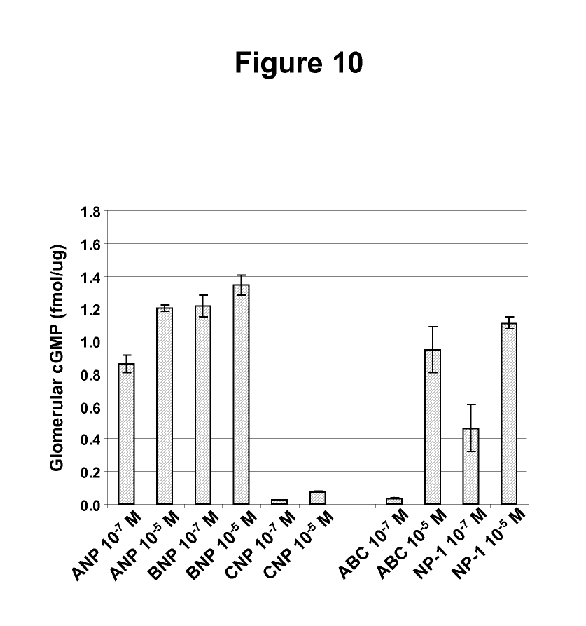

Bolus administration of ABC-NP1 and ABC-NP resulted in diuresis (FIG. 3) and natriuresis (FIG. 4). There was a significant decrease in MAP with ABC-NP1 (p<0.05), but no change with ABC-NP (FIG. 5). In addition, the reduction in RAP and PCWP was significantly greater with ABC-NP1 than with ABC-NP (FIGS. 6 and 7, respectively). cGMP generation in HCF and HAEC was minimal with ABC-NP, and was significantly higher with ABC-NP1 (FIGS. 8 and 9, respectively; p<0.05). In contrast, cGMP generation was similar in isolated canine glomeruli between the two peptides (FIG. 10).

These studies demonstrated that mutation of amino acid residues 15-17 within the CNP ring (from GSM to REA) altered vascular but not renal excretory properties. Hence, a minimal mutation within the CNP ring resulted in a CNP-like peptide that does not have without vascular effects. This ring mutation may be applied to other members of the natriuretic polypeptide family, as described herein.

### Example 2

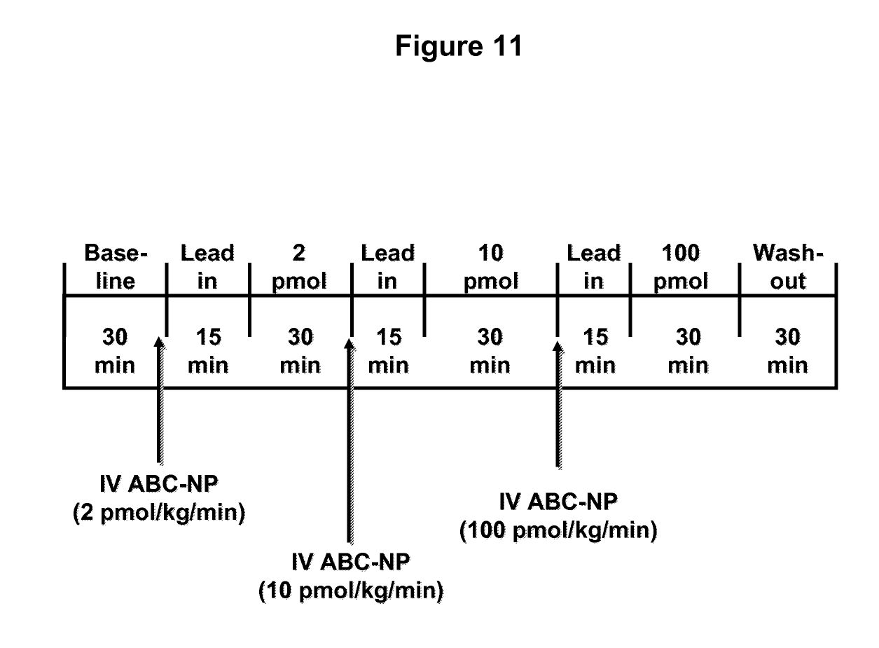

The cardiorenal and humoral actions of intravenous infusions of ABC-NP and ABC-NP1 at 2, 10, and 100 pmol/kg/minute were examined in seven dogs with rapid ventricular pacing-induced overt heart failure with cardiorenal dysfunction (240 bpm for 10 days). As shown in the study protocol depicted in FIG. 11, baseline values for MAP, RAP, PCWP, UNaV, UV, GFR, renal blood flow, urinary cGMP excretion, plasma and cGMP were measured, and animals were then infused with 2 pmol/kg/minute ABC-NP or ABC-NP1 for a lead in period of 15 minutes followed by 30 minutes, then infusion with 10 pmol/kg/minute ABC-NP or ABC-NP1 for a lead in period of 15 minutes followed by 30 minutes, and finally infusion with 100 pmol/kg/minute ABC-NP or ABC-NP1 for a lead in period of 15 minutes followed by 30 minutes, and washout for 30 minutes. Pressure measurements were taken at the end of each NP infusion period, and blood and urine samples also were collected for analysis.

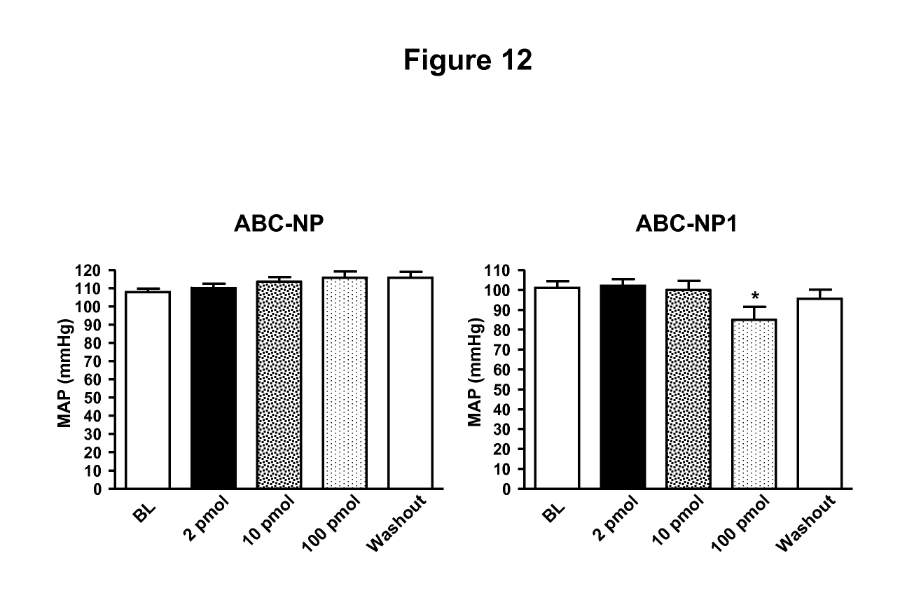

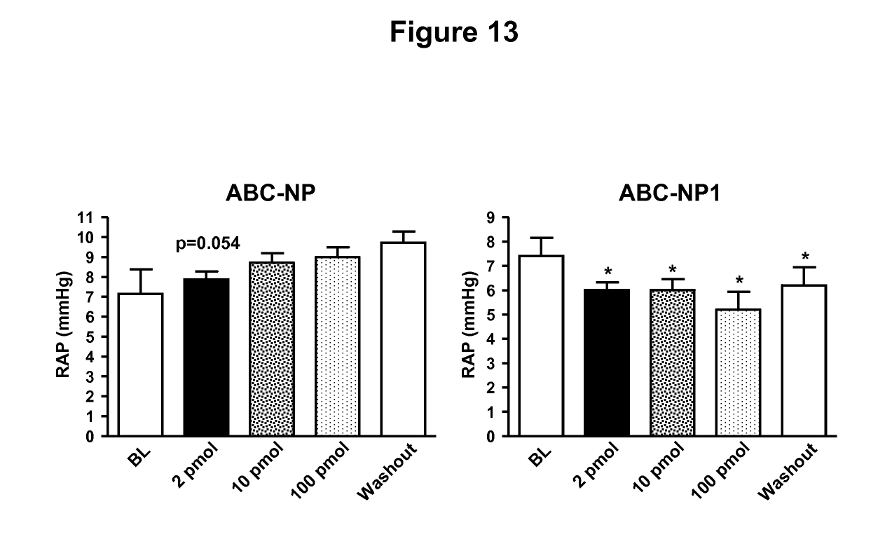

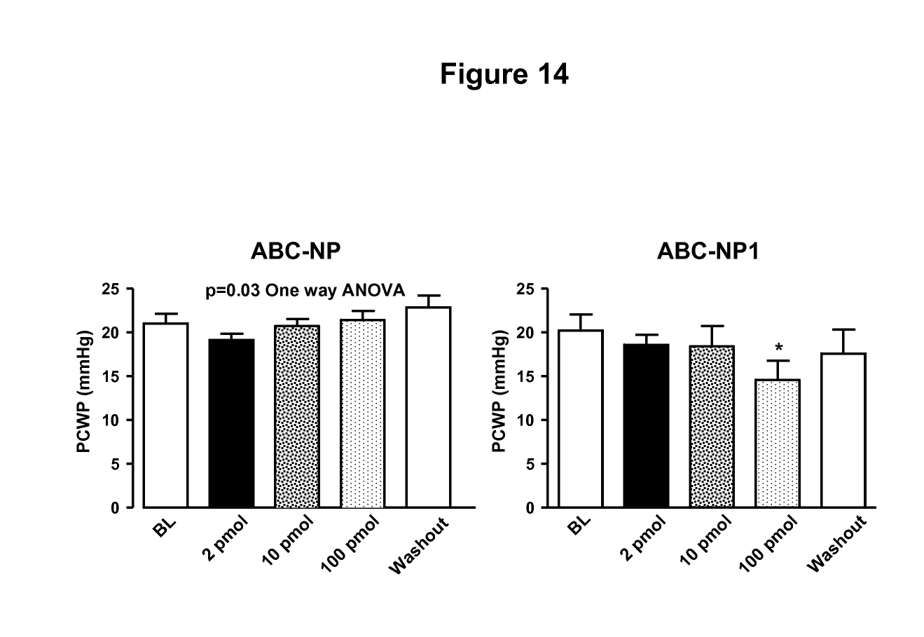

ABC-NP1 significantly reduced MAP and PCWP at the highest dose (FIGS. 12 and 14, respectively), and also significantly reduced RAP at all doses (FIG. 13). ABC-NP had no effect on MAP, and increased RAP. The lowest dose of ABC-NP reduced PCWP as compared to baseline, but the higher doses of ABC-NP did not affect PCWP.

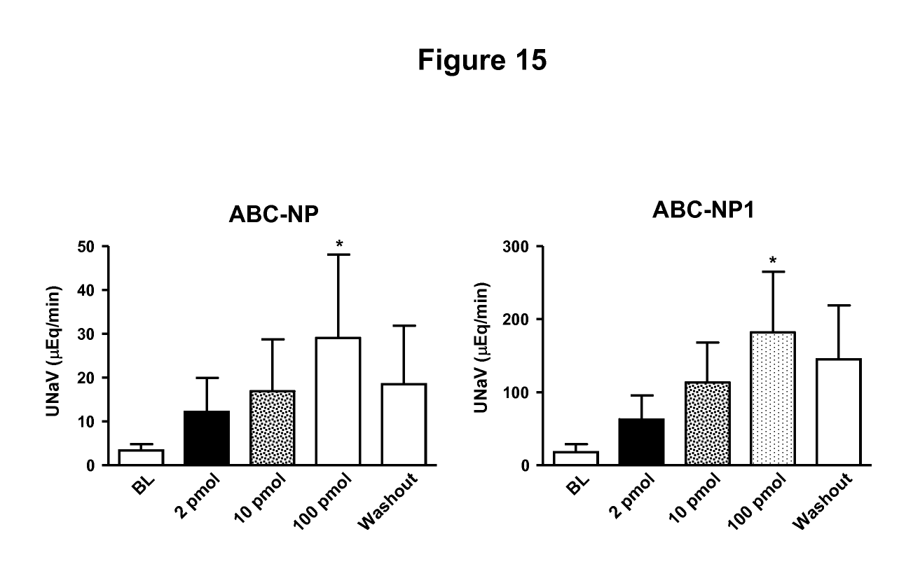

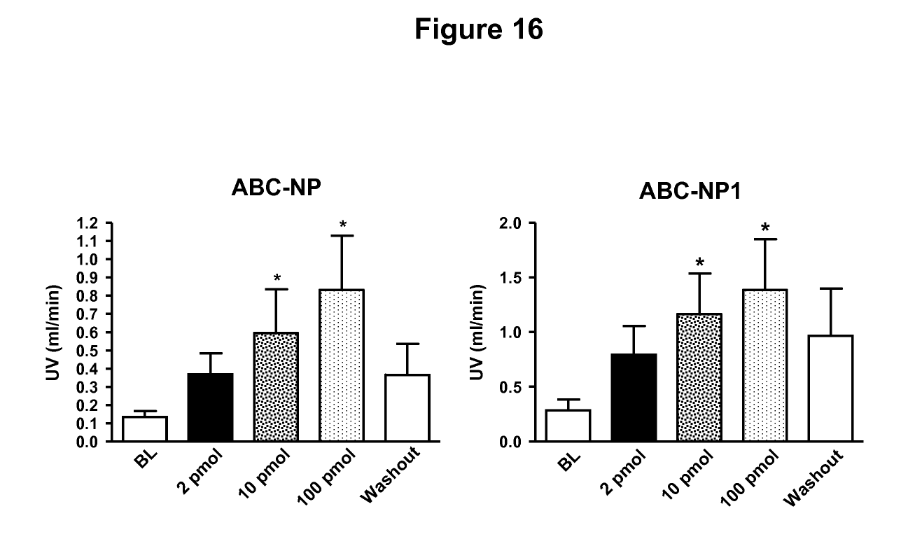

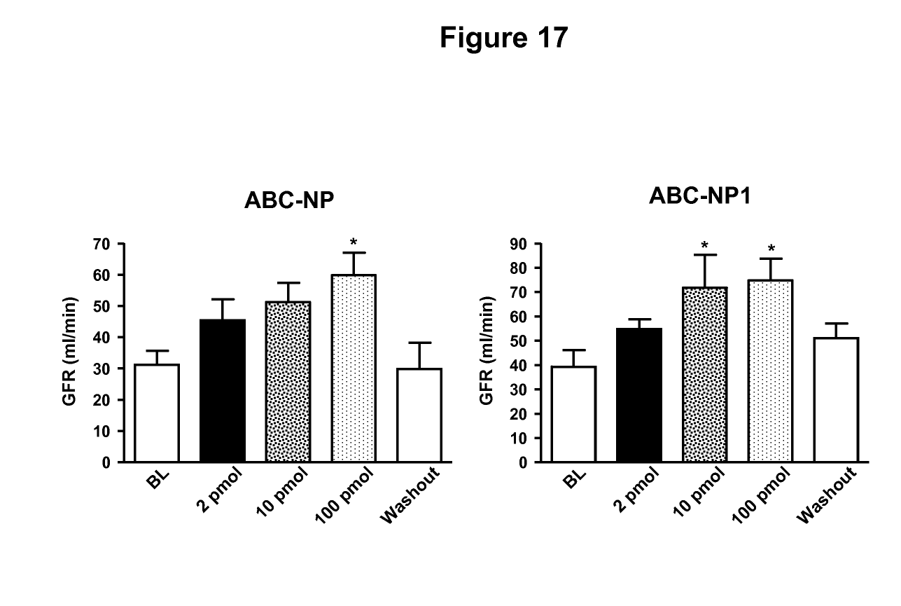

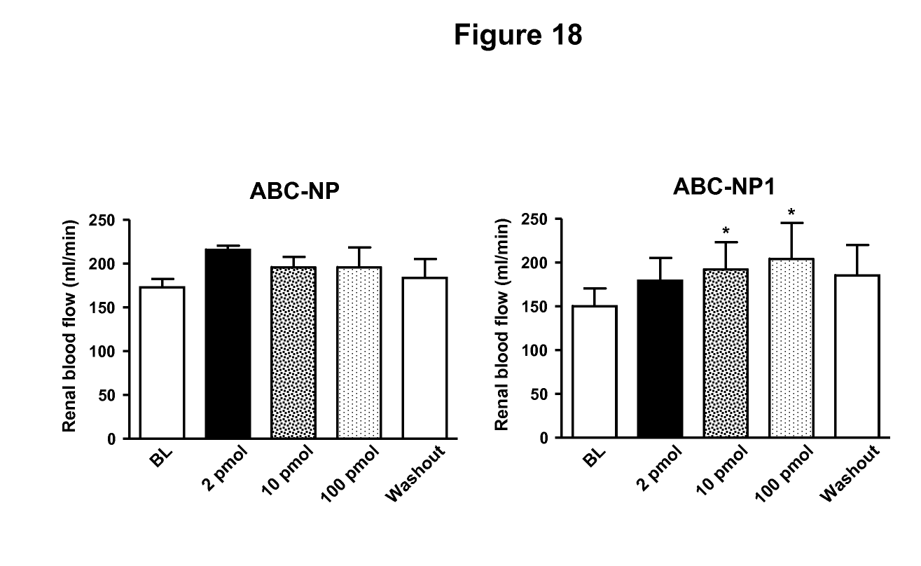

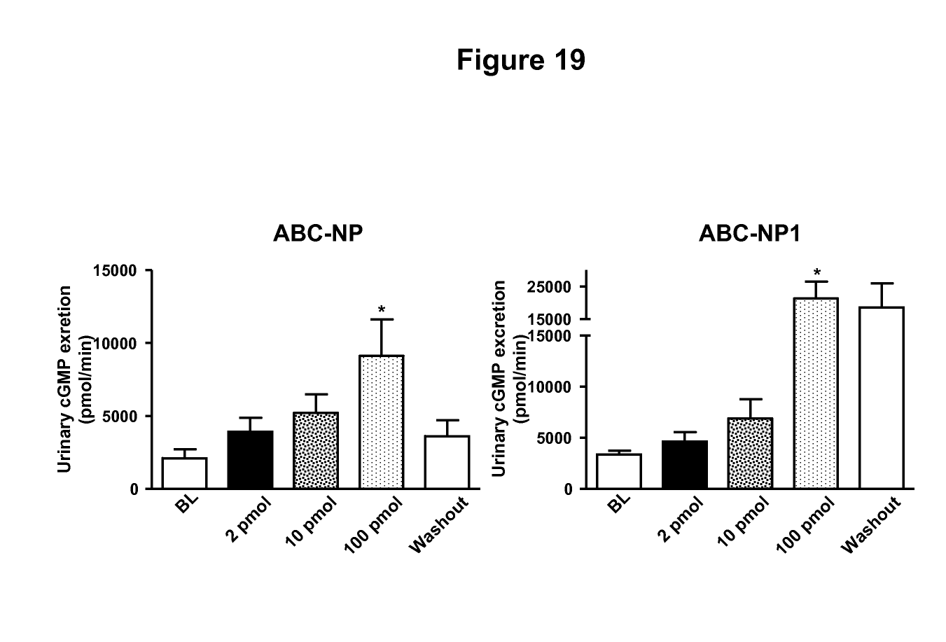

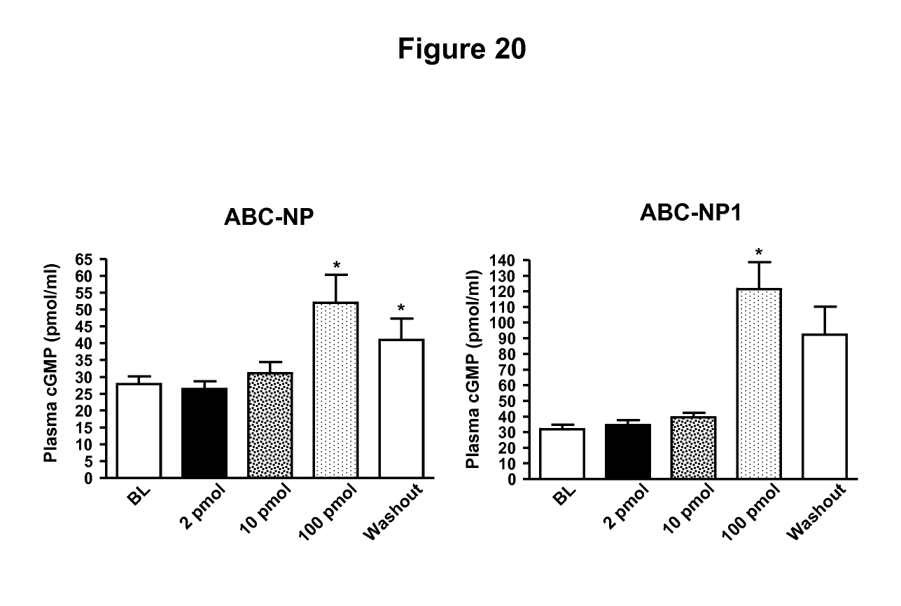

Increasing doses of ABC-NP and ABC-NP1 both had increasing effects on urinary sodium excretion (UNaV) and urine flow (UV), but ABC-NP1 had about a 5- to 6.5-fold greater effect than ABC-NP on UNaV (FIG. 15) and about a 1.5- to 2-fold greater effect than ABC-NP on UV (FIG. 16). The two highest doses of ABC-NP1 resulted in significant increases in glomerular filtration rate (GFR), whereas significance was not achieved with ABC-NP until the highest dose was used (FIG. 17). The two highest doses of ABC-NP1 also gave significant increases in renal blood flow, while ABC-NP had no effect (FIG. 18). The highest dose of both ABC-NP and ABC-NP1 significantly increased urinary cGMP excretion (FIG. 19) and plasma cGMP (FIG. 20), although ABC-NP1 had about a two-fold greater effect than ABC-NP in both cases.

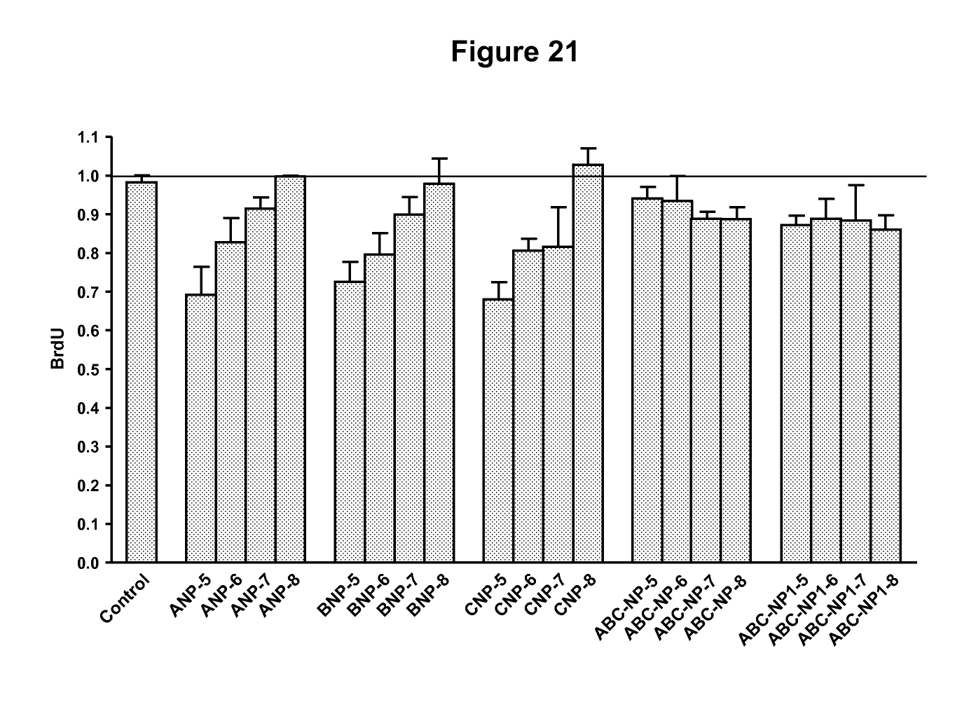

Finally, concentrations of ABC-NP or ABC-NP1 ranging from 10⁻⁵ M to 10⁻⁸ M reduced proliferation of vascular smooth muscle cells in culture, with all concentrations having about the same effect (FIG. 21). In contrast, the effectiveness of ANP, BNP, and CNP decreased as the concentration decreased.

Similar to the studies in Example 1, these studies further demonstrated that mutation of amino acid residues 15-17 within the CNP ring (from GSM to REA) altered vascular but not renal excretory properties. Hence, a minimal mutation within the CNP ring resulted in a CNP-like peptide that does not have without vascular effects. This ring mutation may be applied to other members of the natriuretic polypeptide family, as described herein.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A polypeptide comprising the amino acid sequence set forth in SEQ ID NO:166, wherein the polypeptide comprises an amino acid substitution at each of positions 10, 11, and 12 of SEQ ID NO:166, and wherein the polypeptide does not comprise the sequence set forth in SEQ ID NO:5.

2. The polypeptide of claim 1, wherein the amino acids at positions 10, 11, and 12 of SEQ ID NO:166 are substituted with arginine, glutamic acid, and alanine, respectively.

3. The polypeptide of claim 1, wherein the polypeptide comprises natriuretic activity.

4. The polypeptide of claim 1, wherein the polypeptide comprises the sequence set forth in SEQ ID NO:25.

5. The polypeptide of claim 1, wherein the polypeptide comprises the sequence set forth in SEQ ID NO:30.

6. The polypeptide of claim 1, wherein the polypeptide comprises the sequence set forth in SEQ ID NO:38.

7. The polypeptide of claim 1, wherein the polypeptide comprises the sequence set forth in SEQ ID NO:40.

8. The polypeptide of claim 1, wherein the polypeptide comprises the sequence set forth in SEQ ID NO:43.

9. The polypeptide of claim 1, wherein the polypeptide comprises the sequence set forth in SEQ ID NO:46.

10. The polypeptide of claim 1, wherein the polypeptide comprises the sequence set forth in SEQ ID NO:48.

11. The polypeptide of claim 1, wherein the polypeptide comprises the sequence set forth in SEQ ID NO:51.

12. The polypeptide of claim 1, wherein the polypeptide comprises the sequence set forth in SEQ ID NO:5, 25, 30, 38, 40, 43, 46, 48, or 51, with the proviso that the polypeptide comprises one to three conservative amino acid substitutions.

13. The polypeptide of claim 1, wherein the polypeptide comprises the sequence set forth in SEQ ID NO:5, 25, 30, 38, 40, 43, 46, 48, or 51, with the proviso that the polypeptide comprises one to five conservative amino acid substitutions.

14. The polypeptide of claim 1, wherein the polypeptide is a substantially pure polypeptide.

15. An isolated nucleic acid encoding the polypeptide of claim 1.

16. A vector comprising a nucleic acid encoding the polypeptide of claim 1.

17. A host cell comprising a nucleic acid encoding the polypeptide of claim 1.

18. The host cell of claim 17, wherein the host cell is a eukaryotic host cell.

19. A pharmaceutical composition comprising a pharmaceutically acceptable carrier and the polypeptide of claim 1.

20. A method for increasing natriuretic activity within a mammal, comprising administering a polypeptide of claim 1 to the mammal.

21. A method for treating a mammal having a cardiovascular condition or renal condition, comprising administering to the mammal a polypeptide of claim 1 under conditions wherein the severity of a manifestation of the cardiovascular condition or renal condition is reduced.

22. A method for reducing cardiac remodeling in a subject identified as being in need thereof, comprising administering to the subject the pharmaceutical composition of claim 19, wherein the composition is administered in an amount effective to alter the level of one or more parameters of cardiac remodeling by at least ten percent as compared to the levels of the one or more parameters prior to administering the composition, and wherein the one or more parameters are selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.

23. A method for reducing restenosis in a subject identified as being in need thereof, comprising administering to the subject a restenosis-reducing amount of the pharmaceutical composition of claim 19.
